(19) United States
(12) Patent Application Publication
(10) Pub. No.: US 2026/0168994 A1
(43) Pub. Date: Jun. 18, 2026

(54) POLYMER BRUSH MONOLAYERS FOR SURFACE PASSIVATION FOR BIOMOLECULES AND BIOMOLECULAR RECOGNITION

(71) Applicant: THE REGENTS OF THE UNIVERSITY OF CALIFORNIA, Oakland, CA (US)

(72) Inventors: Beihang YU, El Cerrito, CA (US); Ricardo RUIZ, San Jose, CA (US); Paul D. ASHBY, Alameda, CA (US); Michael D. CONNOLLY, Hercules, CA (US); Grigory TIKHOMIROV, Berkeley, CA (US); Ronald N. ZUCKERMANN, El Cerrito, CA (US)

(21) Appl. No.: 19/532,698

(22) Filed: Feb. 6, 2026

Related U.S. Application Data
(63) Continuation of application No. PCT/US2024/041572, filed on Aug. 8, 2024.
(60) Provisional application No. 63/518,185, filed on Aug. 8, 2023.

(51) Int. Cl. G01N 33/543; G01N 33/53

(52) U.S. Cl. CPC G01N 33/54386; G01N 33/5308

(57) ABSTRACT

The present invention provides for a device comprising a substrate with a monolayer of a poly(methacrylate) (PMA) or polypeptoid polymer

### RELATED PATENT APPLICATIONS

This application claims priority as a continuation application to PCT International Patent Application No. PCT/US2024/041572, filed Aug. 8, 2024, which claims priority to U.S. Provisional Patent Application Ser. No. 63/518,185, filed Aug. 8, 2023, both of which are hereby incorporated by reference.

### STATEMENT OF GOVERNMENTAL SUPPORT

The invention was made with government support under Contract No. DE-AC02-05CH₁₁₂₃₁ awarded by the U.S. Department of Energy. The government has certain rights in the invention.

### FIELD OF THE INVENTION

The present invention is in the field of surface passivation and surface functionalization.

### BACKGROUND OF THE INVENTION

In biotechnology, surface passivation is critical for preventing nonspecific adsorption/interactions of biomacromolecules such as nucleic acids and proteins, where device functions, biophysical assays are tied to specific interactions with biomacromolecules. For DNA nanotechnologies that use folded nucleic acid nanostructures as the functional constructs on surfaces, the surface passivation against nucleic acids is critical for selective and precise placement of nucleic acid nanostructures. However, there are very limited surface passivation strategies with molecules that are simple, robust, and adaptable for different surfaces.

The ability to pattern biomolecules at the nanoscale on Si or other semiconductor substrates at the nanoscale is indispensable to enable devices such as biosensors, biochemical/biophysical assays, next generation sequencers, CMOS-based synthesis, lab-on-a-chip, etc. Bio-compatible nano-patterned surfaces should comprise regions with strong affinity for targeted molecules alternating with passivated regions with no affinity for the biomolecules. Surface passivation prevents nonspecific adsorption of biomolecules to the background, and binding of biomolecules on targeted areas often relies on surface presentation of particular functionalities to induce physisorption or specific recognition. Meeting the simultaneous passivation and functionalization requirement is often challenging. Further, there are very limited material categories with the adaptability to work across different biomolecules typical practice is one strategy works for one particular system.

Harnessing the spatial resolution and positioning accuracy of nanopatterning technology with the programmability and precision of biomacromolecules has the potential to enable technologies for fundamental biophysical research and clinical bioelectronic devices. These devices such as biosensors and disease diagnostic and treatment implants, (1-5) benefit from well-defined surface nanopatterns, offering superior single-molecule resolution and sensitivity compared to homogeneous or micropatterned surfaces. For example, optical nanostructures such as zero-mode waveguides with selective immobilization of enzyme molecules in the confined subdiffraction observation volume enable single-molecule investigation of enzymatic activities at biologically relevant concentrations. (6,7) Nanoscale features on material surfaces (topographical and chemical cues) can program cell adhesion and fate, offering fundamental biological insights into the cell behavior in response to their microenvironment. These insights are crucial for advancing cell culture materials and regenerative medicines including implants and stem cell therapeutics. (8,9) The successful development of bioelectronic or optoelectronic devices interfacing with biological systems with single-molecule resolution will require precise design and customization of surface modification layers with exact nanoscale patterning at the inorganic/bio interface. These surface modification layers, which consist of molecular coatings in direct contact with biological systems, enable the viability of the interface by fulfilling various functions. They can adjust surface hydrophilicity or hydrophobicity, facilitate molecular immobilization or antifouling in specific regions, regulate short-range interactions, and even provide specific molecular recognition.

Fabricating functional and biocompatible nanointerfaces is challenging due to the inherent complexity of biological systems and the need for versatile, customizable surface modification materials that can effectively bridge inorganic and biological surfaces at the nanoscale. These materials must meet three main criteria: (1) versatile interfacial interactions: the surface modification material should enable a broad spectrum of customizable interactions with biomolecules, ranging from repulsion or antifouling properties to various levels of preferential attachment and specific molecular recognition; (2) substrate compatibility: the surface modification should be uniformly thin and applicable to a variety of substrates, including dielectric, semiconductor, and metallic surfaces; (3) compatibility with lithographic processes: the surface modification material should integrate into lithographic flows, and it must withstand common lithographic processes including exposure to organic solvents, resist materials, UV or electron radiation, and high-temperature baking, without compromising its chemical properties and biocompatibility.

Self-assembled monolayers (SAMs) (10-16) and polymer brushes (10,17-29) are perhaps the most commonly used surface modification materials. They have driven many of the recent advances in biosensing and (opto)bioelectronic devices. The molecular structure of SAMs includes a headgroup to attach the molecule to the surface, a backbone that provides structure or self-organization, and a tail group that defines surface energy or provides a site for biomolecular binding. (16) For example, silane-based and thiol-based monolayers are the most common SAM motifs utilized on oxide and noble metal substrates, and a variety of functionalized SAMs are routinely patterned for applications in biological assays and for cell attachment surfaces. (11,14) However, in some cases, SAMs suffer from instabilities or excess reactivities in the attachment chemistry which lead to nonuniform modification layers. (13) Polymer brushes provide a higher level customization with a wider range of chemistry, more controllable surface coverage, and more uniform layer thickness. Yet, as the spatial resolution requirements and complexity of biointerfaces progress, strategies using traditional polymer brushes face challenges. Traditional polymer brushes lack the versatility to incorporate various chemical functionalities required by different biointerfacing needs. Random and block copolymer brushes have been reported, yet in most cases, only two or three monomers are utilized, (24,27) which may not fulfill the requirements of biointerfaces to conveniently tune coupled surface properties such as wettability and surface presence of chemical groups. Polymer brush layers prepared via the “grafting from” method typically produce relatively thick films, often reaching tens of nanometers or more. (27) This thickness limitation is problematic when close proximity of the biomaterial to the inorganic surface is important, as in gated thin-film transistor or thin topographic structures like zero-mode waveguides. (4,7) In summary, there is still a need for versatile, customizable surface modification materials to expand our ability to tune and control a wide variety of interactions at inorganic/bio interfaces that are compatible with high-resolution, high-precision lithographic patterning while maintaining their compatibility with biological systems.

U.S. Pat. No. 10,704,094 (“Multipart Reagents Having Increased Avidity for Polymerase Binding”) discloses multivalent binding compositions, including a particle-nucleotide conjugate having a plurality of copies of a nucleotide attached to the particle.

### SUMMARY OF THE INVENTION

The present invention provides for a device comprising a substrate with a monolayer of a poly(methacrylate) (PMA) or polypeptoid polymer.

In some embodiments, the poly(methacrylate) (PMA), such as poly(methyl methacrylate) (PMMA), brush covalently tethered to the substrate surfaces is used as a simple and robust approach to form a passivation polymer brush monolayer against the adsorption/binding of DNA nanostructures on surfaces. In some embodiments, this strategy using PMMA brushes with corresponding functional end groups for surface attachment, allows surface passivation for different substrates for different devices needed in DNA nanotechnology.

The invention comprises one or more of the following advantages: (1) A simple spin coat-anneal-rinse procedure that yields a covalently tethered PMMA brush monolayer with high coverage and uniformity; (2) Requires small amount of starting material as compared to immersion type coating procedures; (3) End functional PMMA brushes are commercially available and/or easily synthesizable in research/industrial laboratories; (4) The grafted brush passivation layer is temperature and chemically stable, as compared to prior art using hexamethyldisilazane (HMDS); and (5) Different end functional groups are available for PMMA brushes therefore it is easily adaptable beyond oxide substrates.

The invention described here creates a monolayer of tethered poly(methyl methacrylate) (PMMA) brushes on substrate surface for passivation against nucleic acid nanostructures. The PMMA brushes have functional end groups that allow them to covalently tether to substrates and form a monolayer of PMMA brushes. This covalently tethered PMMA brush monolayer robustly passivates the substrate surface when the substrates are incubated with buffer solutions of DNA nanostructures, preventing DNA nanostructures from adsorbing onto the substrates. In some embodiments, the PMMA brushes with a hydroxyl end group reacts with the silanol groups on the silicon oxide surface of silicon wafer substrates, to form a covalently tethered PMMA brush monolayer for surface passivation against DNA origami nanostructures.

In some embodiments, the invention comprises one or more of the following: (1) The invention uses a melt grafting procedure (annealing a thin film of functionalized PMMA molecules deposited on the substrates at elevated temperatures), which requires a small amount of starting material as compared to immersion type coating procedures. (2) End functionalized PMMA brushes are commercially available and/or easily synthesizable in research/industrial laboratories. (3) The covalently tethered passivation monolayer of PMMA brushes is more robust and stable, as compared to prior art using hexamethyldisilane (HMDS) (FIG. 25). (4) Different end functional groups are available for functionalized PMMA, therefore the process to prepare covalently tethered PMMA brush monolayers is easily adaptable beyond oxide substrates (with hydroxyl groups present on substrate surface) as compared to HIMDS, which relies on reacting with the hydroxyl groups on substrate surface.

In some embodiments, the poly(methacrylate) (PMA) has the chemical structure:

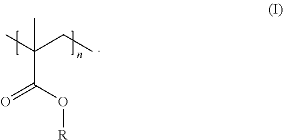

In some embodiments, R is —CH₃, —(CH₂)ₘ—CH₃, —(CH₂)ₘ—CH(CH₃)₂, —(CH₂)ₘ—C(CH₃)₃ or

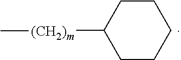

In some embodiments, m is an integer from 0 to 10, from 1 to 10, or from 1 to 5. Suitable poly(methacrylate) subunits are shown in FIG. 34.

In some embodiments, the PMA is poly(methyl methacrylate), poly(ethyl methacrylate), poly(n-propyl methacrylate), poly(isopropyl methacrylate), poly(n-butyl methacrylate), poly(isobutyl methacrylate), poly(cyclohexyl methacrylate), or the like. Exemplary R groups of the PMA are shown in FIGS. 25-28

In some embodiments, the monolayer is a monolayer of functionalized PMMA molecules tethered to a substrate surface to passivate the surface against adsorption of nucleic acid nanostructures.

In some embodiments, the polypeptoid brush, or PMA brush, such as a PMMA brush, is a linear polymer chain comprising a single functional group at one end. In some embodiments, the functional group is a hydroxyl (—OH), primary amine (—NH₂), thiol (—SH), carboxylic acid (—COOH), or dihydroxyphenylalanine (DOPA) functional group.

In some embodiments, the polypeptoid comprises a linear chain of peptoid monomer. In some embodiments, each peptoid monomer independently has a functional group of —(CH₂)ₙOH, —(CH₂)ₙ—O—(CH₂)ₘCH₃, —(CH₂)ₙ—C₆H₅, or —CₙH₂ₘ₊₁, wherein n and m are independently 1, 2, 3, 4, 5, 6, 7, 8, 9, or 10. A Nhb peptoid monomer has a functional group of —(CH₂)₄OH. A Nme peptoid monomer has a functional group of —(CH₂)₂O—CH₃. A Npe peptoid monomer has a functional group of —(CH₂)₂—C₆H₅. A Npm peptoid monomer has a functional group of —CH₂—C₆H₅. A Nib peptoid monomer has a functional group of —CH₂CH(CH₃)₂. A Nbu peptoid monomer has a functional group of —(CH₂)₃CH₃.

In some embodiments, the patterned structure comprises a monolayer of functionalized PMMA polymer molecules tethered to selected patterned areas, producing alternating covered and non-covered regions of tethered PMMA brush molecules.

The present invention provides for a method comprising: (a) a lithographic patterning process (such as optical, electron-beam, nano-imprint, soft lithography, or the like), (b) removing the exposed portions of the PMMA brush not covered by the lithographic resist, and (c) removing the lithographic resist to produce regions that alternate between passivated and non-passivated areas against adsorption of nucleic acid nanostructures.

In some embodiments, the non-passivated regions are features with a lateral dimension in the range of about 10 to 1000 nm.

In some embodiments, the functionalized polymers in the polymethacrylate family are poly(methyl methacrylate), poly(ethyl methacrylate), poly(n-propyl methacrylate), poly(isopropyl methacrylate), poly(n-butyl methacrylate), poly(iso-butyl methacrylate), poly(cyclohexyl methacrylate), and the like. The functionalized polymers can be used to form covalently tethered polymer monolayers to passivate surface against adsorption of nucleic acid nanostructures.

In some embodiments, the technical problems and presumptions overcome include one or more of the following: (1) Surface passivation against nucleic acid nanostructures commonly utilizes a hydrophobic coating of trimethylsilyl via vapor deposition of IMIDS; while here it is demonstrated that a PMMA brush monolayer that is somewhat hydrophilic (water contact angle equal to or more than about 67°) has superior surface passivation against nucleic acid nanostructures. (2) The capability of PMMA brush monolayer to passivate surface against nucleic acid nanostructures is well-maintained after lithographically patterning the PMMA brush monolayer, therefore makes such surface passivation strategy applicable for devices with lithographically pattern features (FIGS. 26-27).

The invention described here creates a monolayer of tethered poly(methyl methacrylate) (PMMA) brushes on substrate surface for passivation against nucleic acid nanostructures. The PMMA brushes have functional end groups that allow them to covalently tether to substrates and form a monolayer of PMMA brushes. This covalently tethered PMMA brush monolayer robustly passivates the substrate surface when the substrates are incubated with buffer solutions of DNA nanostructures, preventing DNA nanostructures from adsorbing onto the substrates. In some embodiments, the PMMA brushes with a hydroxyl end group reacts with the silanol groups on the silicon oxide surface of silicon wafer substrates, to form a covalently tethered PMMA brush monolayer for surface passivation against DNA origami nanostructures.

In some embodiments, the device comprises a monolayer of functionalized PMMA molecules tethered to a substrate surface to passivate the surface against adsorption of a nucleic acid nanostructure.

The following aspects of the invention have been shown: (1) Demonstration of robust surface passivation against DNA nanostructures using covalently tethered PMMA brush monolayers. (2) Demonstration of selective passivation against DNA nanostructures on lithographically patterned surfaces with lateral dimensions below 100 nm.

In some embodiments, individual DNA nanostructure on bound on patterned surfaces, wherein it enabled by selective surface passivation with covalently tethered PMMA brushes.

In some embodiments, the surface is passivated using covalently tethered PMMA brush monolayers against other nucleic acid constructs (such as unfolded or folded nucleic acid strands, such as an oligonucleotide, such as DNA origami).

In some embodiments, the invention can be used as a surface passivation strategy to create surface coatings to prevent non-specific adsorption of DNA nanostructures in devices, in particular for devices that requires stability against harsh conditions and lithographical patterning.

The present invention has one or more of the following advantages: (1) Robustness with better performance, as compared to prior art using HMIDS. (2) Commercial availability of functional PMMA molecules, with different end functional group options, therefore easily adaptable for different substrates that require surface passivation. (3) Simplicity of the preparation method, with minimal starting material required as compared to immersion type procedures. (4) Feasibility of this surface passivation strategy on lithographically patterned surface at high resolution below 100 nm, therefore suitable for applications that use microarrays/nanoarrays and devices with lithographically patterned features.

In some embodiments, the device comprises a material platform based on polypeptoid (N-substituted polyglycines) brushes that can be covalently tethered to substrates, serving as a simple, robust approach to modify surfaces for passivation and functionalization. The polypeptoid brushes are synthesized as sequence-defined, and therefore can be designed on-demand based on the substrates, surface passivation and functionalization requirements.

In some embodiments, the invention has one or more of the following advantages: (1) A simple spin coat-anneal-rinse procedure that yields a covalently tethered polypeptoid brush monolayer with high coverage and uniformity; (2) Requires small amount of starting material as compared to immersion type coating procedures; (3) Different end functional groups are available to easily adapt polypeptoid brushes to different substrates; (4) Different bioconjugation strategies are easily achievable for recognition and binding of different biomolecules; (5) A potential platform to provide universal background passivation using the same polymer backbone scaffold due to its vast monomer chemistry choices.

In some embodiments, the invention uses a class of end-functionalized polypeptoids (N-substituted polyglycines) to create a monolayer of tethered polypeptoid brushes on substrates as surface modification materials. Surfaces modified by polypeptoid brushes are tailored for (1) passivation against nucleic acid nanostructures and/or streptavidin proteins, or ii) binding of nucleic acid nanostructures, or (2) specific binding of streptavidin by biotin-streptavidin interactions. In some embodiments, the device uses polypeptoid brushes with a hydroxyl end group that reacts with the silanol groups on the silicon oxide surface, to form a covalently tethered polypeptoid brush monolayer for the above selective passivation and functionalization for binding purposes.

In some embodiments, the invention comprises one or more of the following: (2) The invention uses a melt grafting procedure (annealing a thin film of functionalized polypeptoids deposited on the substrates at elevated temperatures), which requires a small amount of starting material as compared to immersion type coating procedures. (2) The large monomer library of polypeptoids enable incorporation of desired anchoring functionalities to easily adapt to different substrates beyond oxide substrate surfaces. (3) The large monomer library of polypeptoids enable incorporation of desired binding functionalities through bioconjugation to specifically recognize and bind to corresponding biomolecules. (4) With the demonstrated compatibility with lithographical patterning and tailorable monomer design, surface nanopatterns presenting multiple different biomolecule binding areas are possible using tethered polypeptoid brushes as surface modification layers.

In some embodiments, the monolayer of functionalized polypeptoid (N-substituted polyglycines) molecules tethered to a substrate surface is used as a surface modification material for passivation or functionalization for binding to biomolecules.

In some embodiments, the polypeptoid brush is a linear polymer chain comprising a single functional group at the other end where the functional group may be a biotin (for specific binding to streptavidin or neutravidin), alkyne/azide/thiol/ene (for click chemistry), or a peptide having a biological function, such as binding or adhesion to another compound, molecule, or object, such as a cell. An example of a peptide is the RGD (Arg-Gly-Asp) peptide which is capable of cell adhesion. Another example of a peptide is the His₆ (His-His-His-His-His-His) peptide which is capable of binding nickel. In some embodiments, the polypeptoid brush comprises an end peptoid monomer having a substrate grafting functionality. In some embodiments, the end peptoid monomer comprises a functional group comprising one or more —OH. In some embodiments, the end peptoid monomer has a functional group of —(CH₂)ₙOH, wherein n is 1, 2, 3, 4, 5, 6, 7, 8, 9, or 10. In some embodiments, the linear polymer chain comprises one or more Nhb, Nme, Npe, Npm, Nib, and/or Nbu peptoid monomers.

In some embodiments, the polypeptoid brushes are monodisperse, sequence-defined polymer chains, with each monomer of the polymer chain precisely defined.

In some embodiments, the patterned structure comprises a monolayer of correspondingly functionalized polypeptoid molecules tethered to selected patterned areas, producing regions with surface passivation and regions with active binding properties.

In some embodiments, the structure comprises the monolayer and (a) a lithographic patterning process (optical, electron-beam, nano-imprint, soft lithography, or the like), (b) removing the exposed portions of the polypeptoid brush not covered by the lithographic resist, (c) removing the lithographic resist to produce regions that alternate between areas with and without tethered polypeptoid brushes, and (d) tether a second type of functionalized polypeptoids to the exposed regions to form a patterned structure with two different polypeptoid brushes tethered to selected areas.

In some embodiments, the patterned structure comprises a feature with a lateral dimension in the range of about 10 to 1000 nm.

In some embodiments, the patterned structure selectively binds to biomacromolecules to regions with about 10 to 1000 nm lateral dimension, with minimal nonspecific adsorption of biomacromolecules on the non-binding regions (such as a passivated background).

In some embodiments, the invention overcomes one or more of the following: (1) Surface passivation in biotechnology predominately use poly(ethylene glycol) (PEG) based materials, while here it is demonstrated that a polypeptoid scaffold, with appropriate monomer chemistry choice (backbone constant, side group variable), can be designed to have superior surface passivation against nucleic acid nanostructures and/or streptavidin protein. (2) As a new surface modification material, it is demonstrated that its thermal stability (heating at elevated temperatures for grafting reactions), compatibility with lithographical patterning processes involving background electron-beam exposure, organic solvents during development and rinsing, reactive ion etching, sonication (without noticeable brush detachment), and ability to stay contamination free after patterning with an electron-beam resist. (3) With the potential interdigitation of the two types of polymer brushes, one is able to devise ways to maintain the passivation and binding properties in the respective regions.

The following aspects of the invention have been shown: (1) Demonstration of robust surface passivation against or active binding of nucleic acid nanostructures using covalently tethered polypeptoid brush monolayers. (2) Demonstration of robust surface passivation against or active binding of streptavidin proteins using covalently tethered polypeptoid brush monolayers. (3) Demonstration of selective passivation against and specific binding of streptavidin proteins on lithographically patterned surfaces with binding regions of dimension equal to or less than about 200 nm, or equal to or less than about 100 nm.

In some embodiments, the covalently tethered polypeptoid brush monolayers provide surface passivation against specific proteins.

In some embodiments, there is selective passivation against and specific binding of other biomolecules on lithographically patterned surfaces, with binding regions functionalized with covalently tethered polypeptoid brushes that have the corresponding recognition and binding group towards the target biomolecules.

In some embodiments, the invention can be used for: DNA nanotechnology, bioprinting, protein sequencing, or biotechnology devices. In some embodiments, the invention can be used for devices or products that require selective surface passivation against and binding of biomolecules. The invention can be used as a surface passivation or functionalization strategy to create surface coatings to either prevent non-specific adsorption of biomolecules, or actively bind to biomolecules in devices, in particular for devices that requires stability against harsh conditions and lithographical patterning.

In some embodiments, the invention has one or more of the following advantages: (1) Simplicity of the preparation method, with minimal starting material required as compared to immersion type procedures. (2) Feasibility of this surface passivation strategy on lithographically patterned surface at high resolution below 100 nm or 200 nm, therefore suitable for applications that use microarrays/nanoarrays and devices with lithographically patterned features. (3) On-demand design of the polypeptoid brushes depending on the particular biomolecules to passivate against or to bind.

In some embodiments, the substrate is SiO₂, Si/SiO₂, metal, glass, flexible polymer substrates, composites, or a hybrid thereof.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. (a) Preparation of grafted polypeptoid brush monolayers on Si substrates via the “grafting to” approach with a hydroxyl (—OH) group as the substrate grafting functionality incorporated on the first monomer at the C-terminus. (b) Polypeptoids synthesized for Example 1.

FIG. 2. (a) ATR-FTIR and XPS N is spectra of prepared polypeptoid brush monolayers on Si substrates. The presence of an IR band of ˜1670 cm⁻¹ for polypeptoid amide C═O stretching and a N is peak between 390 and 400 eV confirms successful grafting of —OH functionalized polypeptoids. (b) Topography and PiFM (mapped at 1648 cm⁻¹, corresponding to the amide C═O stretching of polypeptoids) characterization indicates good surface coverage of the substrate by the grafted polypeptoid brush monolayer with a smooth top surface.

FIG. 3. Surface hydrophilicity of Si substrates, as evidenced by different static water contact angles, is modified accordingly by the grafted polypeptoid brush monolayers, depending on the monomer chemistry and monomer composition. See FIG. 1 (panel b) for chemical structures of the polypeptoids.

FIG. 4. Si substrates modified with different polypeptoid brush monolayers exhibit tunable affinity from passivation to preferential attachment of DNA origami nanostructures.

FIG. 5. Immobilization of streptavidin via specific biotin-streptavidin interactions at different binding densities on surfaces, achieved by controlling surface biotin group density through grafting biotin-PP1 and PP1 mixtures (relative concentration of biotin-PP1 in PP1, c_{b}) onto Si substrates.

FIG. 6. (a) Schematic of the lithographic patterning workflow to pattern a polypeptoid brush monolayer grafted on Si substrate: (i) electron-beam lithography to pattern the spin-coated PMMA resist layer on top of the brush monolayer, (ii) reactive ion etching with oxygen plasma to transfer the pattern into the underlying polypeptoid brush monolayer, and (iii) stripping the PMMA resist and reannealing the patterned polypeptoid brush monolayer. (b) AFM topography images of generated line-space patterns (pitch=170 nm) with different line widths, from which the height profiles show the polypeptoid brush monolayer is ˜1 nm in thickness.

FIG. 7. (a) Combined PiFM image mapped at 1658 and 1113 cm⁻¹, corresponding to amide C═O stretching of polypeptoids and Si—O—Si stretching of SiO₂, respectively, demonstrates the successful electron-beam lithographic patterning of polypeptoid brush monolayers grafted on Si substrates. The PiF-IR signal intensity ratios of 1658 cm⁻¹/1113 cm⁻¹ at locations on Si (SiO₂) trenches (locations 2, 4, 6, and 8) are near zero; i.e., trenches are clean with unobservable polypeptoid residues. (b) From bottom to top: averaged PiF-IR spectrum of 6 locations on a pristine polypeptoid brush monolayer before electron-beam lithography, averaged PiF-IR spectrum of locations 1, 3, 5, and 7 on the polypeptoid brush lines generated by electron-beam lithography, averaged PiF-IR spectrum of locations 2, 4, 6, and 8 on Si (SiO₂) trenches post-electron-beam lithographic patterning. The similar PiF-IR spectra of the pristine polypeptoid brush monolayer and nanopatterned polypeptoid brush lines indicate that the chemical characteristics of this polypeptoid brush-based system are preserved through the lithographic workflow.

FIG. 8. Surface chemical contrast nanopatterns consisting of alternating lines of two polymer brushes are generated by patterning a first polymer brush monolayer (e.g., PMMA-OH or PS-OH) with electron-beam lithography and then backfilling a second polymer (polypeptoid-OH) to graft onto the exposed Si (SiO₂) surface. The combined PiFM images mapped at wavenumbers corresponding to the characteristic bands of each polymer (polypeptoid, 1664 cm⁻¹; PMMA, 1723 cm⁻¹; PS, 697 cm⁻¹) confirm the chemical characteristics of the generated surface nanopatterns.

FIG. 9. (a) Selective binding of DNA origami (61 nm×52 nm×8 nm cuboid nanostructure with an aperture) on surface nanopatterns of PMMA-PP3, where the circular patches modified by PP3 brushes exhibit high binding affinity toward DNA origami, and the rest of the surface is passivated by PMMA brushes against DNA origami. (b) Selective binding of streptavidin (100 nM in 1×PBS buffer) on surface nanopatterns of (biotin-PP1)-PP1, where the circular patches are modified with biotin-PP1 brushes for specific biotin-streptavidin interactions and the rest of the surface is passivated by nonbiotinylated PP1 brushes against nonspecific binding of streptavidin.

FIG. 10. UPLC-MS (C18 column) of PP1, Nme₂₀Nhb, theoretical molecular weight: 2448.8 g mol⁻¹.

FIG. 11. UPLC-MS of PP2 (C18 column), (NmeNpe)₁₀Nhb, theoretical molecular weight: 2909.6 g mol⁻¹.

FIG. 12. UPLC-MS of PP3 (C18 column), Nme₅(Npm₃Npe)₃Npm₃Nhb, theoretical molecular weight: 2971.6 g mol⁻¹.

FIG. 13. UPLC-MS (C4 column) of PP4, (NpeNpm₃)₅Nhb, theoretical molecular weight: 3159.9 g mol⁻¹.

FIG. 14. UPLC-MS of PP5 (C18 column), Nme₅(Nbu₃Nib)₃Nbu₃Nhb, theoretical molecular weight: 2419.3 g mol⁻¹.

FIG. 15. UPLC-MS of biotin-PP1 (C18 column), biotin-Nme₂₀Nhb, theoretical molecular weight: 2675.1 g mol⁻¹.

FIG. 16. Potential esterification of the —OH group on the first monomer with TFA during cleavage (peak at 3.27 min observed in LC trace, with +96 g mol⁻¹ as compared to the theoretical molecular weight of PP1), which fully reverse after ˜two days in ACN/H₂O solution.

FIG. 17. Polymer/Polypeptoid Thin Film and Monolayer Characterization. Topography and PiFM images of prepared polypeptoid brush monolayers on Si substrates with —OH vs. —COOH functionalized polypeptoids (Nme₅(Npm₃Npe)₃Npm₃Nhb vs. Nme₅(Npm₃Npe)₃Npm₃Nce).

FIG. 18. Surface Chemical Contrast Nanopattern Characterization. IR PiFM of PMMA-polypeptoid nanopattern.

FIG. 19. IR PiFM of PS-polypeptoid nanopattern.

FIG. 20. DNA Origami and Streptavidin Immobilization on Polymer Brush Modified Surfaces and Chemical Contrast Nanopatterns. Top: Schematic of the DNA origami nanostructure (from tilibit nanosystems) and morphology of deposited DNA origami nanostructures on bare Si substrates. Bottom: DNA origami binding density on bare Si (3 nM) vs. on PP3 brush monolayer and PP4 brush monolayer modified Si substrates (1 nM).

FIG. 21. Passivation test against non-specific binding streptavidin (1 μM in 1×PBS buffer) on different polypeptoid brush and PMMA brush modified Si substrates.

FIG. 22. Different streptavidin binding density (quantified by streptavidin fractional surface coverage, φ) is achieved by controlling surface biotin group density through grafting biotin-PP1 and PP1 mixtures (relative concentration of biotin-PP1 in PP1, c_{b}). Binarization of AFM images were performed by thresholding with either clustering with the “KMedoids” method (c_{b}=0.05, 0.1, 0.2), or the “MinimumError” method (c_{b}=0, 1) when the two height populations corresponding to streptavidin surface and polymer brush surface have minimum overlap in the height histogram.

FIG. 23. Backfill test with the biotinylated polypeptoid (biotin-PP1) brush, which demonstrates sufficient brush interpenetration happens during the backfill step, and the interpenetrated biotinylated polypeptoid brushes bind large amounts of streptavidin proteins.

FIG. 24. (a) Tert-butyldimethylsilyl (tBDMS) protection 4-amino-1-butanol. (b) Biotinylation of polypeptoids at the N-terminus.

FIG. 25. Comparison between using HMDS and PMMA brush for surface passivation of Si wafer substrates against DNA origami nanostructures (without surface passivation, a bare Si wafer binds to DNA origami nanostructures). Si wafer with vapor deposited HMDS does not have whole DNA origami (square shapes as seen on bare Si wafer), yet there are debris of materials on the surface after incubation of the DNA origami buffer solutions (it is also reported that the formed trimethylsilyl layer from HMDS undergoes hydrolyzation at basic pHs>9 or after long periods of soaking in incubation buffer1). Si wafer with grafted PMMA brush monolayer stays clean after incubation of DNA origami buffer solutions.

FIG. 26. Demonstration of selective binding of DNA origami nanostructures on lithographically patterned substrate surfaces, where the DNA origami nanostructures selectively bind to exposed Si (SiO2) surface, or polypeptoid brushes grafted surface, enabled by passivating the rest of the surface with PMMA brushes. (a) 500 nm circular features, (b) alternating line patterns with 170 nm pitch.

FIG. 27. Surface patterns that bind individual DNA origami nanostructures at designated locations. Lithographical features (on a background passivated with PMMA brushes) with dimensions comparable to individual DNA origami nanostructures are created, and make the features have preferential binding to DNA origami nanostructures to enable the selectivity.

FIG. 28. Exemplary R groups of the PMA.

FIG. 29. Overview of the polypeptoid brush platform. a) Functionalized polypeptoids are synthesized as sequence-defined using solid phase synthesis. For the polypeptoids to covalently attach to the substrate surface (native SiO2 layer on Si wafer), here is introduced a hydroxy function on the first monomer as the side chain. b) The simple, robust, scalable workflow to prepare polypeptoid brush monomers on substrates.

FIG. 30. The chemical structures of the five polypeptoids described and used in the embodiments.

FIG. 31. With the five different polypeptoids, a monolayer of each polypeptoid brush is tethered to Si wafer substrates. The surface energy of the substrate is modified correspondingly with these about 1 nm thick polypeptoid brush monolayers, as indicated by the water contact angle in blue. The modified surfaces also display different binding affinity towards DNA origami, with Polypeptoid 1 and Polypeptoid 5 display minimal affinity to DNA origami, rendering them as excellent passivation surface modification layers against DNA origami, despite their distinct surface hydrophilicity. Polypeptoid 3 and Polypeptoid 4 are found to have high binding affinity towards DNA origami.

FIG. 32. Among the different polypeptoids, Polypeptoid 1 is identified as a good surface passivation material against streptavidin.

FIG. 33. a) Biotinylation of polypeptoids is achieved by reacting D-biotin with the free N-terminus of polypeptoid chains on resin as the last coupling step in the solid-phase synthesis. b) Surface nanopatterns with patches consisting of tethered biotin-Polypeptoid 1 brush for specific binding of streptavidin and background consisting of tethered Polypeptoid 1 for passivation again streptavidin is created and demonstrated to selectively bind to streptavidin via biotin-streptavidin interactions in the circular patches with diameters down to 100 nm.

FIG. 34. Various suitable poly(methacrylate) subunits.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” and “˜” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

References cited herein:
(1) Zhirnov, V. 2018 Semiconductor Synthetic Biology Roadmap; SRC, NIST, 2018.
(2) Bathe, M.; Chrisey, L. A.; Herr, D. J. C.; Lin, Q.; Rasic, D.; Woolley, A. T.; Zadegan, R. M.; Zhirnov, V. V. Roadmap on Biological Pathways for Electronic Nanofabrication and Materials. Nano Futures 2019, 3, No. 012001.
(3) Tian, B.; Xu, S.; Rogers, J. A.; Cestellos-Blanco, S.; Yang, P.; Carvalho-de-Souza, J. L.; Bezanilla, F.; Liu, J.; Bao, Z.; Hjort, M.; Cao, Y.; Melosh, N.; Lanzani, G.; Benfenati, F.; Galli, G.; Gygi, F.; Kautz, R.; Gorodetsky, A. A.; Kim, S. S.; Lu, T. K.; Anikeeva, P.; Cifra, M.; Krivosudsky, O.; Havelka, D.; Jiang, Y. Roadmap on Semiconductor-Cell Biointerfaces. Phys. Biol. 2018, 15, No. 031002.
(4) Jiang, Y.; Tian, B. Inorganic Semiconductor Biointerfaces. Nat. Rev. Mater. 2018, 3, 473-490.
(5) Craighead, H. Future Lab-on-a-Chip Technologies for Interrogating Individual Molecules. Nature 2006, 442, 387-93.
(6) Korlach, J.; Marks, P. J.; Cicero, R. L.; Gray, J. J.; Murphy, D. L.; Roitman, D. B.; Pham, T. T.; Otto, G. A.; Foquet, M.; Turner, S. W. Selective Aluminum Passivation for Targeted Immobilization of Single DNA Polymerase Molecules in Zero-Mode Waveguide Nanostructures. Proc. Natl. Acad. Sci. U.S.A. 2008, 105, 1176-81.
(7) Levene, M. J.; Korlach, J.; Turner, S. W.; Foquet, M.; Craighead, H. G.; Webb, W. W. Zero-Mode Waveguides for Single-Molecule Analysis at High Concentrations. Science 2003, 299, 682-686.
(8) Dalby, M. J.; Gadegaard, N.; Oreffo, R. O. Harnessing Nanotopography and Integrin-Matrix Interactions to Influence Stem Cell Fate. Nat. Mater. 2014, 13, 558-69.
(9) Zhang, W.; Yang, Y.; Cui, B. New Perspectives on the Roles of Nanoscale Surface Topography in Modulating Intracellular Signaling. Curr. Opin. Solid State Mater. Sci. 2021, 25, 100873.
(10) Senaratne, W.; Andruzzi, L.; Ober, C. K. Self-Assembled Monolayers and Polymer Brushes in Biotechnology: Current Applications and Future Perspectives. Biomacromolecules 2005, 6, 2427-2448.
(11) Lahiri, J.; Ostuni, E.; Whitesides, G. M. Patterning Ligands on Reactive Sams by Microcontact Printing. Langmuir 1999, 15, 2055-2060.
(12) Kumar, A.; Biebuyck, H. A.; Whitesides, G. M. Patterning Self-Assembled Monolayers: Applications in Materials Science. Langmuir 1994, 10, 1498-1511.
(13) Haensch, C.; Hoeppener, S.; Schubert, U. S. Chemical Modification of Self-Assembled Silane Based Monolayers by Surface Reactions. Chem. Soc. Rev. 2010, 39, 2323-34.
(14) Smith, R. K.; Lewis, P. A.; Weiss, P. S. Patterning Self-Assembled Monolayers. Prog. Surf. Sci. 2004, 75, 1-68.
(15) Wang, L.; Schubert, U. S.; Hoeppener, S. Surface Chemical Reactions on Self-Assembled Silane Based Monolayers. Chem. Soc. Rev. 2021, 50, 6507-6540.
(16) Casalini, S.; Bortolotti, C. A.; Leonardi, F.; Biscarini, F. Self-Assembled Monolayers in Organic Electronics. Chem. Soc. Rev. 2017, 46, 40-71.
(17) Husemann, M.; Morrison, M.; Benoit, D.; Frommer, J.; Mate, C. M.; Hinsberg, W. D.; Hedrick, J. L.; Hawker, C. J. Manipulation of Surface Properties by Patterning of Covalently Bound Polymer Brushes. J. Am. Chem. Soc. 2000, 122, 1844-1845.
(18) Dong, R.; Krishnan, S.; Baird, B. A.; Lindau, M.; Ober, C. K. Patterned Biofunctional Poly(Acrylic Acid) Brushes on Silicon Surfaces. Biomacromolecules 2007, 8, 3082-3092.
(19) Liu, C.-C.; Han, E.; Onses, M. S.; Thode, C. J.; Ji, S.; Gopalan, P.; Nealey, P. F. Fabrication of Lithographically Defined Chemically Patterned Polymer Brushes and Mats. Macromolecules 2011, 44, 1876-1885.
(20) Xue, C.; Yonet-Tanyeri, N.; Brouette, N.; Sferrazza, M.; Braun, P. V.; Leckband, D. E. Protein Adsorption on Poly(N-Isopropylacry-lamide) Brushes: Dependence on Grafting Density and Chain Collapse. Langmuir 2011, 27, 8810-8.
(21) Chen, T.; Amin, I.; Jordan, R. Patterned Polymer Brushes. Chem. Soc. Rev. 2012, 41, 3280-96.
(22) Frenzel, R.; Hohne, S.; Hanzelmann, C.; Schmidt, T.; Winkler, R.; Drechsler, A.; Bittrich, E.; Eichhorn, K. J.; Uhlmann, P. Tunable Hydrophilic or Amphiphilic Coatings: A “Reactive Layer Stack” Approach. ACS Appl. Mater. Interfaces 2015, 7, 12355-66.
(23) Kim, M.; Schmitt, S.; Choi, J.; Krutty, J.; Gopalan, P. From Self-Assembled Monolayers to Coatings: Advances in the Synthesis and Nanobio Applications of Polymer Brushes. Polymers (Basel) 2015, 7, 1346-1378.
(24) Chen, W.-L.; Cordero, R.; Tran, H.; Ober, C. K. 50th Anniversary Perspective: Polymer Brushes: Novel Surfaces for Future Materials. Macromolecules 2017, 50, 4089-4113.
(25) Chen, W.-L.; Menzel, M.; Prucker, O.; Wang, E.; Ober, C. K.; Rü he, J. Morphology of Nanostructured Polymer Brushes Dependent on Production and Treatment. Macromolecules 2017, 50, 4715-4724.
(26) Yuksekdag, Y. N.; Gevrek, T. N.; Sanyal, A. Diels-Alder “Clickable” Polymer Brushes: A Versatile Catalyst-Free Conjugation Platform. ACS Macro Lett. 2017, 6, 415-420.
(27) Zoppe, J. O.; Ataman, N. C.; Mocny, P.; Wang, J.; Moraes, J.; Klok, H. A. Surface-Initiated Controlled Radical Polymerization: State-of-the-Art, Opportunities, and Challenges in Surface and Interface Engineering with Polymer Brushes. Chem. Rev. 2017, 117, 1105-1318.
(28) Bueno-Alejo, C. J.; Santana Vega, M.; Chaplin, A. K.; Farrow, C.; Axer, A.; Burley, G. A.; Dominguez, C.; Kara, H.; Paschalis, V.; Tubasum, S.; Eperon, I. C.; Clark, A. W.; Hudson, A. J. Surface Passivation with a Perfluoroalkane Brush Improves the Precision of Single-Molecule Measurements. ACS Appl. Mater. Interfaces 2022, 14, 49604-16.
(29) Wang, Y.-M.; Kilosi, A.; Halahovets, Y.; Romanenko, I.; Slaby, J.; Homola, J.; Svoboda, J.; de los Santos Pereira, A.; Pop-Georgievski, O. Grafting Density and Antifouling Properties of Poly[N-(2-Hydroxypropyl) Methacrylamide] Brushes Prepared by “Grafting to” and “Grafting From. Polym. Chem. 2022, 13, 3815-3826.
(30) Zuckermann, R. N.; Kerr, J. M.; Kent, S. B. H.; Moos, W. H. Efficient Method for the Preparation of Peptoids [Oligo(NSubstituted Glycines)] by Submonomer Solid-Phase Synthesis. J. Am. Chem. Soc. 1992, 114, 10646-10647.
(31) Knight, A. S.; Zhou, E. Y.; Francis, M. B.; Zuckermann, R. N. Sequence Programmable Peptoid Polymers for Diverse Materials Applications. Adv. Mater. 2015, 27, 5665-91.
(32) Fetsch, C.; Luxenhofer, R. Thermal Properties of Aliphatic Polypeptoids. Polymers (Basel) 2013, 5, 112-127.
(33) Miller, S. M.; Simon, R. J.; Ng, S.; Zuckermann, R. N.; Kerr, J. M.; Moos, W. H. Proteolytic Studies of Homologous Peptide and NSubstituted Glycine Peptoid Oligomers. Bioorg. Med. Chem. Lett. 1994, 4, 2657-2662.
(34) Miller, S. M.; Simon, R. J.; Ng, S.; Zuckermann, R. N.; Kerr, J. M.; Moos, W. H. Comparison of the Proteolytic Susceptibilities of Homologous L-Amino Acid, D-Amino Acid, and N-Substituted Glycine Peptide and Peptoid Oligomers. Drug Dev. Res. 1995, 35, 20-32.
(35) Zuckermann, R. N. Peptoid Origins. Biopolymers 2011, 96, 545-55.
(36) Culf, A. S.; Ouellette, R. J. Solid-Phase Synthesis of NSubstituted Glycine Oligomers (Alpha-Peptoids) and Derivatives. Molecules 2010, 15, 5282-5335.
(37) Zhuravlev, L. T. The Surface Chemistry of Amorphous Silica. Zhuravlev Model. Colloids Surf. A Physicochem. Eng. Asp. 2000, 173, 1-38.
(38) Luo, T.; Zhang, R.; Zeng, W.-W.; Zhou, C.; Yang, X.; Ren, Z. Alkoxylation Reaction of Alcohol on Silica Surfaces Studied by Sum Frequency Vibrational Spectroscopy. J. Phys. Chem. C 2021, 125, 8638-8646.
(39) Brittain, W. J.; Minko, S. A Structural Definition of Polymer Brushes. J. Polym. Sci. A Polym. Chem. 2007, 45, 3505-3512.
(40) Zhao, B.; Brittain, W. J. Polymer Brushes: Surface-Immobilized Macromolecules. Prog. Polym. Sci. 2000, 25, 677-710.
(41) Rosales, A. M.; Mumen, H. K.; Kline, S. R.; Zuckermann, R. N.; Segalman, R. A. Determination of the Persistence Length of Helical and Non-Helical Polypeptoids in Solution. Soft Matter 2012, 8, 3673.
(42) Yu, B.; Danielsen, S. P. O.; Yang, K. C.; Ho, R. M.; Walker, L. M.; Segalman, R. A. Insensitivity of Sterically Defined Helical Chain Conformations to Solvent Quality in Dilute Solution. ACS Macro Lett. 2020, 9, 849-854.
(43) Nowak, D.; Morrison, W.; Wickramasinghe, H. K.; Jahng, J.; Potma, E.; Wan, L.; Ruiz, R.; Albrecht Thomas, R.; Schmidt, K.; Frommer, J.; Sanders Daniel, P.; Park, S. Nanoscale Chemical Imaging by Photoinduced Force Microscopy. Sci. Adv. 2016, 2, No. e1501571.
(44) Jahng, J.; Potma, E. O.; Lee, E. S. Nanoscale Spectroscopic Origins of Photoinduced Tip-Sample Force in the Midinfrared. Proc. Natl. Acad. Sci. U.S.A. 2019, 116, 26359.
(45) Almajhadi, M. A.; Uddin, S. M. A.; Wickramasinghe, H. K. Observation of Nanoscale Opto-Mechanical Molecular Damping as the Origin of Spectroscopic Contrast in Photo Induced Force Microscopy. Nat. Commun. 2020, 11, 5691.
(46) Zhao, X.; Zhao, W.; Zheng, X.; Rafailovich, M. H.; Sokolov, J.; Schwarz, S. A.; Pudensi, M. A. A.; Russell, T. P.; Kumar, S. K.; Fetters, L. J. Configuration of Grafted Polystyrene Chains in the Melt: Temperature and Concentration Dependence. Phys. Rev. Lett. 1992, 69, 776-779.
(47) Keddie, J. L.; Jones, R. A. L. Glass Transition Behavior in UltraThin Polystyrene Films. Isr. J. Chem. 1995, 35, 21-26.
(48) Mansky, P.; Liu, Y.; Huang, E.; Russell, T. P.; Hawker, C. Controlling Polymer-Surface Interactions with Random Copolymer Brushes. Science 1997, 275, 1458-1460.
(49) Liu, G.; Ji, S.; Stuen, K. O.; Craig, G. S. W.; Nealey, P. F.; Himpsel, F. J. Modification of a Polystyrene Brush Layer by Insertion of Poly(Methyl Methacrylate) Molecules. J. Vac. Sci. Technol. B 2009, 27, 3038-3042.
(50) Wan, L.; Ruiz, R.; Gao, H.; Albrecht, T. R. Self-Registered Self Assembly of Block Copolymers. ACS Nano 2017, 11, 7666-7673.
(51) Laus, M.; Chiarcos, R.; Gianotti, V.; Antonioli, D.; Spamacci, K.; Munao, G.; Milano, G.; De Nicola, A.; Perego, M. Evidence of Mechanochemical Control in “Grafting to” Reactions of Hydroxy Terminated Statistical Copolymers. Macromolecules 2021, 54, 499-508.
(52) Chang, B. S.; Loo, W. S.; Yu, B.; Dhuey, S.; Wan, L.; Nealey, P. F.; Ruiz, R. Sequential Brush Grafting for Chemically and Dimensionally Tolerant Directed Self-Assembly of Block Copolymers. ACS Appl. Mater. Interfaces 2023, 15, 2020-2029.
(53) Schmidt, S. W.; Christ, T.; Glockner, C.; Beyer, M. K.; Clausen Schaumann, H. Simple Coupling Chemistry Linking Carboxyl Containing Organic Molecules to Silicon Oxide Surfaces under Acidic Conditions. Langmuir 2010, 26, 15333-8.
(54) Kurihara, K.; Mizukami, M. Hydrogen-Bonded Surface Macroclusters of Carboxylic Acid on Silica in Cyclohexane. Proc. Jpn. Acad. Ser. B Phys. Biol. Sci. 2001, 77, 115-120.
(55) Zhang, L.; Mizukami, M.; Kurihara, K. Preparation of Poly(Acrylic Acid) Nano-Films by in-Situ Polymerization of Acrylic Acid Macroclusters on Silicon Oxide Surfaces. Macromol. Symp. 2008, 270, 40-47.
(56) Rosales, A. M.; Mumen, H. K.; Zuckermann, R. N.; Segalman, R. A. Control of Crystallization and Melting Behavior in Sequence Specific Polypeptoids. Macromolecules 2010, 43, 5627-5636.
(57) Sun, J.; Teran, A. A.; Liao, X.; Balsara, N. P.; Zuckermann, R. N. Crystallization in Sequence-Defined Peptoid Diblock Copolymers Induced by Microphase Separation. J. Am. Chem. Soc. 2014, 136, 2070-7.
(58) van Zoelen, W.; Buss, H. G.; Ellebracht, N. C.; Lynd, N. A.; Fischer, D. A.; Finlay, J.; Hill, S.; Callow, M. E.; Callow, J. A.; Kramer, E. J.; Zuckermann, R. N.; Segalman, R. A. Sequence of Hydrophobic and Hydrophilic Residues in Amphiphilic Polymer Coatings Affects Surface Structure and Marine Antifouling/Fouling Release Properties. ACS Macro Lett. 2014, 3, 364-368.
(59) Gimenez, D.; Zhou, G.; Hurley, M. F. D.; Aguilar, J. A.; Voelz, V. A.; Cobb, S. L. Fluorinated Aromatic Monomers as Building Blocks to Control Alpha-Peptoid Conformation and Structure. J. Am. Chem. Soc. 2019, 141, 3430-3434.
(60) Kershner, R. J.; Bozano, L. D.; Micheel, C. M.; Hung, A. M.; Fomof, A. R.; Cha, J. N.; Rettner, C. T.; Bersani, M.; Frommer, J.; Rothemund, P. W.; Wallraff, G. M. Placement and Orientation of Individual DNA Shapes on Lithographically Patterned Surfaces. Nat. Nanotechnol. 2009, 4, 557-61.
(61) Gopinath, A.; Rothemund, P. W. K. Optimized Assembly and Covalent Coupling of Single-Molecule DNA Origami Nanoarrays. ACS Nano 2014, 8, 12030-12040.
(62) Gopinath, A.; Thachuk, C.; Mitskovets, A.; Atwater, H. A.; Kirkpatrick, D.; Rothemund, P. W. K. Absolute and Arbitrary Orientation of Single-Molecule Shapes. Science 2021, 371, 1.
(63) Shetty, R. M.; Brady, S. R.; Rothemund, P. W. K.; Hariadi, R. F.; Gopinath, A. Bench-Top Fabrication of Single-Molecule Nanoarrays by DNA Origami Placement. ACS Nano 2021, 15, 11441-11450.
(64) Aghebat Rafat, A.; Pirzer, T.; Scheible, M. B.; Kostina, A.; Simmel, F. C. Surface-Assisted Large-Scale Ordering of DNA Origami Tiles. Angew. Chem., Int. Ed. Engl. 2014, 53, 7665-8.
(65) Statz, A. R.; Meagher, R. J.; Barron, A. E.; Messersmith, P. B. New Peptidomimetic Polymers for Antifouling Surfaces. J. Am. Chem. Soc. 2005, 127, 7972-7973
(66) Statz, A. R.; Barron, A. E.; Messersmith, P. B. Protein, Cell and Bacterial Fouling Resistance of Polypeptoid-Modified Surfaces: Effect of Side-Chain Chemistry. Soft Matter 2008, 4, 131-139.
(67) Ham, H. O.; Park, S. H.; Kurutz, J. W.; Szleifer, I. G.; Messersmith, P. B. Antifouling Glycocalyx-Mimetic Peptoids. J. Am. Chem. Soc. 2013, 135, 13015-22.
(68) Ruiz, R.; Kang, H.; Detcheverry, F. A.; Dobisz, E.; Kercher, D. S.; Albrecht, T. R.; de Pablo, J. J.; Nealey, P. F. Density Multiplication and Improved Lithography by Directed Block Copolymer Assembly. Science 2008, 321, 936-939.
(69) Liu, C.-C.; Ramirez-Hernindez, A.; Han, E.; Craig, G. S. W.; Tada, Y.; Yoshida, H.; Kang, H.; Ji, S.; Gopalan, P.; de Pablo, J. J.; Nealey, P. F. Chemical Patterns for Directed Self-Assembly of Lamellae-Forming Block Copolymers with Density Multiplication of Features. Macromolecules 2013, 46, 1415-1424.
(70) Rosales, A. M.; McCulloch, B. L.; Zuckermann, R. N.; Segalman, R. A. Tunable Phase Behavior of Polystyrene-Polypeptoid Block Copolymers. Macromolecules 2012, 45, 6027-6035.
(71) Patterson, A. L.; Yu, B.; Danielsen, S. P. O.; Davidson, E. C.; Fredrickson, G. H.; Segalman, R. A. Monomer Sequence Effects on Interfacial Width and Mixing in Self-Assembled Diblock Copolymers. Macromolecules 2020, 53, 3262-3272.
(72) Ospina, V. M.; Chiarcos, R.; Antonioli, D.; Gianotti, V.; Laus, M.; Kuschlan, S.; Wiemer, C.; Perego, M. Brush Layers of Bioinspired Polypeptoids for Deterministic Doping of Semiconductors. ACS Appl. Electron. Mater. 2022, 4, 6029-6037.
(73) Wu, T.; Efimenko, K.; Vlcek, \hat{P}.; Ŝubr, V.; Genzer, J. Formation and Properties of Anchored Polymers with a Gradual Variation of Grafting Densities on Flat Substrates. Macromolecules 2003, 36, 2448-2453.
(74) Dumont, E. L. P.; Belmas, H.; Hess, H. Observing the Mushroom-to-Brush Transition for Kinesin Proteins. Langmuir 2013, 29, 15142-15145.
(75) Lee, K.-B.; Park, S.-J.; Mirkin, C. A.; Smith, J. C.; Mrksich, M. Protein Nanoarrays Generated by Dip-Pen Nanolithography. Science 2002, 295, 1702-1705.
(76) Schvartzman, M.; Palma, M.; Sable, J.; Abramson, J.; Hu, X.; Sheetz, M. P.; Wind, S. J. Nanolithographic Control of the Spatial Organization of Cellular Adhesion Receptors at the Single-Molecule Level. Nano Lett. 2011, 11, 1306-1312.
(77) Toledo, E.; Le Saux, G.; Edri, A.; Li, L.; Rosenberg, M.; Keidar, Y.; Bhingardive, V.; Radinsky, O.; Hadad, U.; Di Primo, C.; Buffeteau, T.; Smith, A.-S.; Porgador, A.; Schvartzman, M. Molecular-Scale Spatio-Chemical Control of the Activating-Inhibitory Signal Integration in Nk Cells. Sci. Adv. 2021, 7, No. eabc1640.
(78) Feng, H.; Dolejsi, M.; Zhu, N.; Yim, S.; Loo, W.; Ma, P.; Zhou, C.; Craig, G. S. W.; Chen, W.; Wan, L.; Ruiz, R.; de Pablo, J. J.; Rowan, S. J.; Nealey, P. F. Optimized Design of Block Copolymers with Covarying Properties for Nanolithography. Nat. Mater. 2022, 21, 1426-1433.
(79) Lee, M.; Kang, D. K.; Yang, H. K.; Park, K. H.; Choe, S. Y.; Kang, C.; Chang, S. I.; Han, M. H.; Kang, I. C. Protein Nanoarray on Prolinker Surface Constructed by Atomic Force Microscopy Dip-Pen Nanolithography for Analysis of Protein Interaction. Proteomics 2006, 6, 1094-103.
(80) Scheideler, O. J.; Yang, C.; Kozminsky, M.; Mosher, K. I.; Falcon-Banchs, R.; Ciminelli, E. C.; Bremer, A. W.; Chem, S. A.; Schaffer, D. V.; Sohn, L. L. Recapitulating Complex Biological Signaling Environments Using a Multiplexed, DNA-Patterning Approach. Sci. Adv. 2020, 6, No. eaay5696.
(81) Reed, B. D.; Meyer, M. J.; Abramzon, V.; Ad, O.; Ad, O.; Adcock, P.; Ahmad, F. R.; Alppay, G.; Ball, J. A.; Beach, J.; Belhachemi, D.; Bellofiore, A.; Bellos, M.; Beltrin, J. F.; Betts, A.; Bhuiya, M. W.; Blacklock, K.; Boer, R.; Boisvert, D.; Brault, N. D.; et al. Real-Time Dynamic Single-Molecule Protein Sequencing on an Integrated Semiconductor Device. Science 2022, 378, 186-192.
(82) Shrestha, P.; Yang, D.; Tomov, T. E.; MacDonald, J. I.; Ward, A.; Bergal, H. T.; Krieg, E.; Cabi, S.; Luo, Y.; Nathwani, B.; JohnsonBuck, A.; Shih, W. M.; Wong, W. P. Single-Molecule Mechanical Fingerprinting with DNA Nanoswitch Calipers. Nat. Nanotechnol. 2021, 16, 1362-1370.
(83) Connolly, M. D.; Xuan, S.; Molchanova, N.; Zuckermann, R. N. Chapter Eight—Submonomer Synthesis of Sequence Defined Peptoids with Diverse Side-Chains. In Methods in Enzymology; Petersson, E. J., Ed.; Academic Press, 2021; Vol. 656, pp 241-270.
(84) Owens, D. K.; Wendt, R. C. Estimation of the Surface Free Energy of Polymers. J. Appl. Polym. Sci. 1969, 13, 1741-1747.
(85) Kaelble, D. H.; Uy, K. C. A Reinterpretation of Organic Liquid Polytetrafluoroethylene Surface Interactions. J. Adhes. 1970, 2, 50-60.
(86) Kaelble, D. H. Dispersion-Polar Surface Tension Properties of Organic Solids. J. Adhes. 1970, 2, 66-81.
(87) Zuckermann, R. N.; Martin, E. J.; Spellmeyer, D. C.; Stauber, G. B.; Shoemaker, K. R.; Kerr, J. M.; Figliozzi, G. M.; Goff, D. A.; Siani, M. A.; Simon, R. J.; Banville, S. C.; Brown, E. G.; Wang, L.; Richter, L. S.; Moos, W. H., Discovery of Nanomolar Ligands for 7-Transmembrane G-Protein-Coupled Receptors from a Diverse N-(Substituted)glycine Peptoid Library. J. Med. Chem. 1994, 37, 2678-2685.

### Example 1

### Nanopatterned Monolayers of Bioinspired, Sequence-Defined Polypeptoid Brushes for Semiconductor/Bio Interfaces

The ability to control and manipulate semiconductor/bio interfaces is essential to enable biological nanofabrication pathways and bioelectronic devices. Traditional surface functionalization methods, such as self-assembled monolayers (SAMs), provide limited customization for these interfaces. Polymer brushes offer a wider range of chemistries, but choices that maintain compatibility with both lithographic patterning and biological systems are scarce. Here, we developed a class of bioinspired, sequence-defined polymers, i.e., polypeptoids, as tailored polymer brushes for surface modification of semiconductor substrates. Polypeptoids featuring a terminal hydroxyl (—OH) group are designed and synthesized for efficient melt grafting onto the native oxide layer of Si substrates, forming ultrathin (˜1 nm) monolayers. By programming monomer chemistry, our polypeptoid brush platform offers versatile surface modification, including adjustments to surface energy, passivation, preferential biomolecule attachment, and specific biomolecule binding. Importantly, the polypeptoid brush monolayers remain compatible with electron-beam lithographic patterning and retain their chemical characteristics even under harsh lithographic conditions. Electron-beam lithography is used over polypeptoid brushes to generate highly precise, binary nanoscale patterns with localized functionality for the selective immobilization (or passivation) of biomacromolecules, such as DNA origami or streptavidin, onto addressable arrays. This surface modification strategy with bioinspired, sequence-defined polypeptoid brushes enables monomer-level control over surface properties with a large parameter space of monomer chemistry and sequence and therefore is a highly versatile platform to precisely engineer semiconductor/bio interfaces for bioelectronics applications.

Here, we introduce a family of surface modification materials based on bioinspired, sequence-defined polypeptoid brushes, which offer an ideal means to bridge the gap between synthetic and biomaterials. There is also a need to bridge the gap between small molecule SAMs that have precise molecular structures and higher molecular weight yet disperse polymers. Polypeptoids, or poly(N-substituted glycine)s, (30,31) share similarities with polypeptides but offer better solubility and processability in a range of organic solvents and increased resistance to thermal and protease degradation. (32-35) They can be precisely synthesized with defined sequences for tailored molecular structures and functionalities. Furthermore, polypeptoids feature highly versatile and diverse side chains, (31,36) which can range from enabling compatibility with inorganic materials and processes to ensuring compatibility and stability with biomaterials. We designed and synthesized polypeptoids with a terminal hydroxyl (—OH) group that reacts with the activated surface with silanol groups (37,38) to graft the polymer onto Si substrates and form ˜1 nm thick tethered brush monolayers, which enable efficient modification of the surface properties. This work is organized as follows: In the first section, we detail the design and structure of five polypeptoids of different compositions and sequences, along with the process for creating uniform brush monolayers grafted on Si substrates. The second section focuses on the versatility of the polypeptoid brush platform, highlighting its capability to customize and fine-tune a wide range of interfacial interactions. This includes (1) modulating surface energy, demonstrated by achieving a range of water contact angles; (2) achieving passivation and selective immobilization of biomolecules through various short-range interactions, particularly in relation to DNA origami nanostructures; (3) enabling specific recognition and binding of biomolecules at surfaces via biotin-streptavidin interactions. In the final section of the paper, we demonstrate compatibility of the polypeptoid brushes with lithographic processes to generate nanoscale patterns offering high-resolution chemical contrast and diverse chemical functionalities. We utilized these nanopatterned surfaces to achieve highly selective and localized immobilization of DNA origami nanostructures or streptavidin proteins.

### RESULTS AND DISCUSSION

### Grafting Polypeptoid-OH onto Si Substrates as Surface Modification Layers

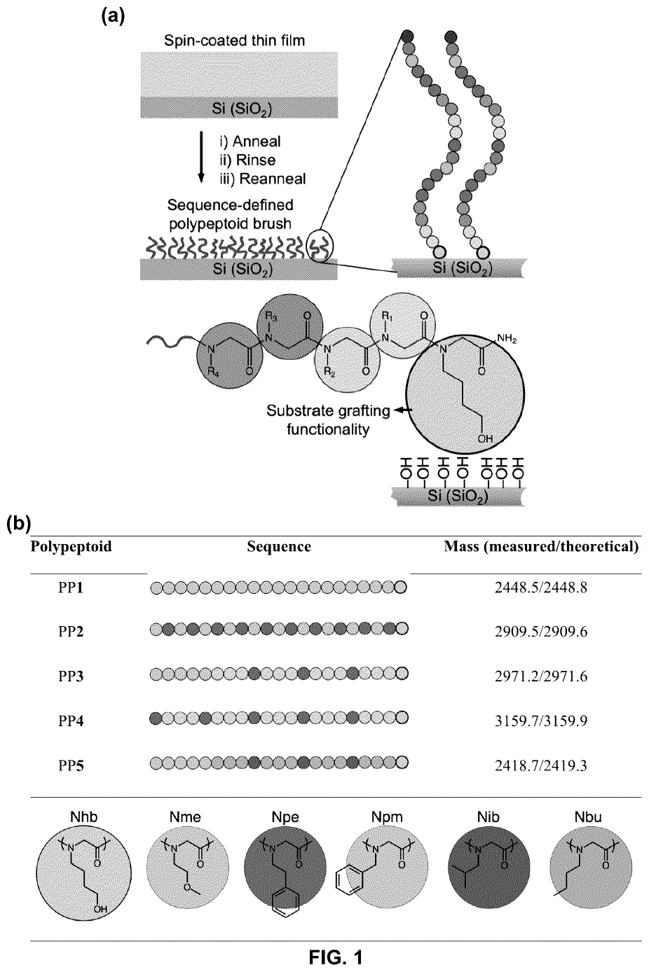

Surface-tethered polymer brushes created through the “grafting from” method are known for forming denser monolayers compared to the “grafting to” method. (24,29,39,40) However, the latter eliminates the need for in situ polymerization and offers broader substrate compatibility while still delivering effective surface modification as long as coverage is uniform at relevant molecular scales. Here, we choose the convenience of the “grafting to” method. The polypeptoids feature a hydroxyl (—OH) group on the side chain of the first monomer at the C-terminus (FIG. 1), providing strong binding to oxide surfaces like activated SiO₂ with silanol groups. (37,38) Five hydroxyl-terminated polypeptoid 21-mers (PP1 through PP5, FIG. 1, panel b) were designed and synthesized with a combination of polar and nonpolar monomers to adjust hydrophilicity and other properties impacting processability, such as solubility in organic solvents and crystallization inhibition for spin coating, as well as compatibility with liquid chromatography characterization.

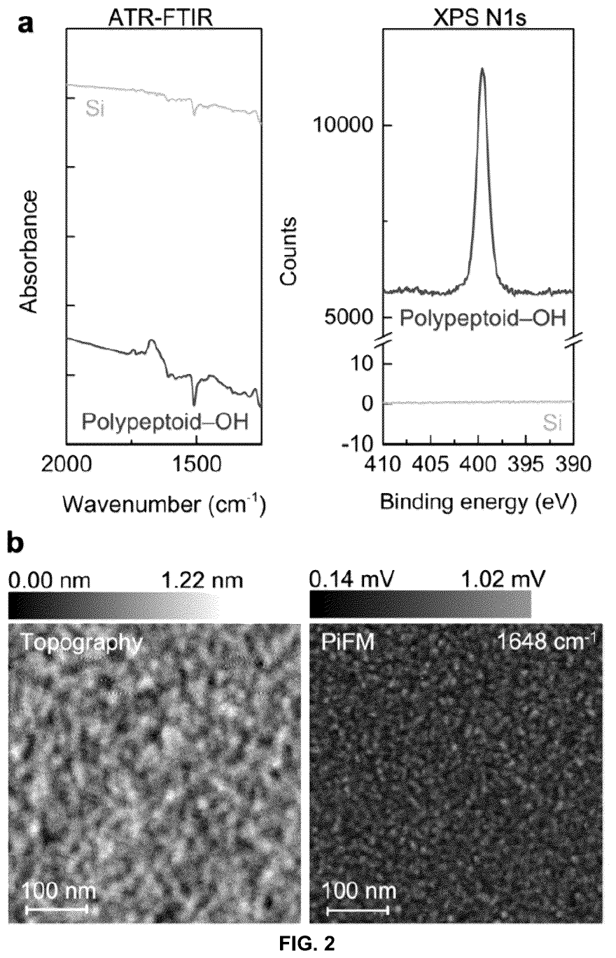

The polypeptoid 21-mers used in this study have molecular weights between 2400 and 3200 g mol⁻¹ (FIG. 1, panel b), which would have a radius of gyration (R_{g}) of ˜1 nm based on previous small-angle neutron scattering (SANS) measurements of similar polypeptoids in near-θ conditions. (41,42) With the potentially very thin brush monolayers, multiple surface characterization techniques are employed to confirm the surface coverage of Si substrates with grafted polypeptoids. With attenuated total reflectance-Fourier transform infrared (ATR-FTIR) spectroscopy and X-ray photoelectron spectroscopy (XPS), the presence of grafted polypeptoids on substrates is confirmed by the amide C═O stretching peak at ˜1670 cm⁻¹ in ATR-FTIR, and the N is peak between 390 and 400 eV in XPS (FIG. 2). Yet, these two macroscopic characterization techniques do not provide direct evidence on whether homogeneous modification of the substrate surface on nanoscopic length scales is achieved.

In order to examine the surface coverage at nanoscale resolution, we further use infrared photoinduced force microscopy (IR PiFM) to directly visualize the surface coverage of Si substrates by grafted polypeptoids. IR PiFM is a nanoscale microscopy and spectroscopy technique that measures photoinduced thermal response and polarizability of samples in the near field by detecting the force between the tip and the sample. This technique enables simultaneous spatial mapping of topographical and chemical information from two mechanical eigenmode resonances of the cantilever at the resolution of an atomic force microscope (AFM). (43-45) The prepared polypeptoid brush monolayers are topographically smooth, with a root-mean-square roughness of R_{q}≈0.2 nm, comparable to bare Si substrates. Importantly, the chemical map collected simultaneously at 1648 cm⁻¹ (characteristic band of polypeptoid backbone amide C═O stretching) indicates that the substrate is well covered with a grafted polypeptoid brush monolayer (FIG. 2). These results indicate the feasibility of achieving uniform coverage and, notably, uniform chemical modification using the “grafting to” method, at least down to the resolution of an AFM (a few nanometers in lateral resolution), despite the lower grafting density (a) characteristic of the “grafting to” approach. Further estimation of the value of a will be discussed later in the manuscript. While both carboxylic acid (—COOH) and hydroxyl (—OH) functionalities have been reported in literature as functional groups for grafting polymers to the native oxide layer of Si substrates under melt grafting conditions, (19,46-52) here, we found that, under the same melt grafting conditions, i.e., annealing polypeptoid thin films spin coated on Si substrates at 180° C. for 30 min, only the —OH functionalized polypeptoids efficiently graft onto Si substrates, while the —COOH functionalized polypeptoids only form discrete aggregates on the equivalently treated substrates (FIG. 17). Similar trends in grafting efficiency between PS-OH and PS-COOH and PMMA-OH and PMMA-COOH are observed under the same melt grafting conditions (Table 1). Condensation reactions between carboxylic acids and free silanol groups have been reported under acetic conditions (pH=2) with a 0-15% coupling efficiency. (53) Without the presence of an acid in solution, molecules with carboxylic acid groups may only physisorb on silicon oxide surfaces via hydrogen bonds. (54,55) We suspect that our conditions with thermal annealing are insufficient for the —COOH functionalized polymers to form stable bonds with surface silanol groups as compared to —OH functionalized polymers.

### Customization in Surface Modification Enabled by Polypeptoid Brushes

The ultrathin polypeptoid brush monolayers are excellent surface modification materials because they can be used to controllably tune the surface properties of Si substrates. Using polypeptoids with a terminal hydroxyl group, we demonstrate that, through designing monomer chemistry and composition, different levels of complexity in modifying surface properties can be achieved: from the simplest surface energy, to affinity toward biomolecules from passivation to preferential attachment, and further to specific binding of biomolecules.

### Surface Hydrophilicity and Surface Free Energy

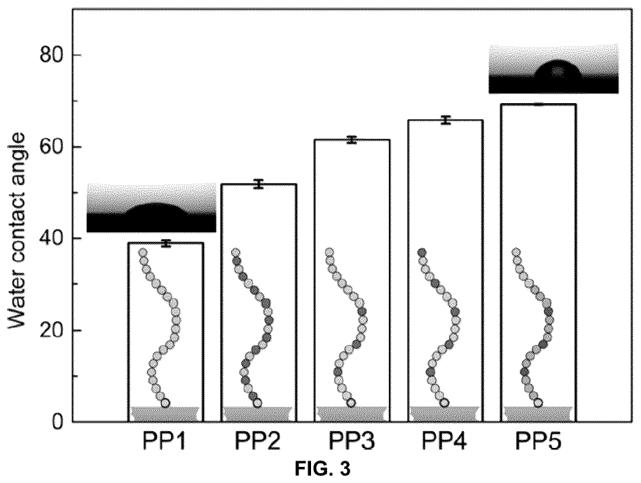

With the grafted brush monolayers of PP1-5, the surface hydrophilicity of Si substrates can be modified accordingly as evidenced by the static water contact angle ranging from 38.9±0.70 to 69.2±0.1° (FIG. 3). With the amide backbone, polypeptoids are polar in nature, yet by introducing side chains of different polarities, the overall polarity of the polymer can be tuned. It is expected that polar side chains (e.g., the methoxyethyl group) would lead to more hydrophilic surfaces and surfaces with a higher polar contribution in the surface free energy. It is also expected that nonpolar aromatic and alkyl groups would lower the overall polarity of the polypeptoid. Indeed, polypeptoid brushes with ≥50% of nonpolar side chains lead to much higher water contact angles and a very small polar contribution in the surface free energy, as compared to PP1 modified surfaces (Table 2). Here, we note the polypeptoid brush modified surfaces are not hydrophobic; however, it is possible to further expand the tunable range of surface hydrophilicity and surface free energy with grafted polypeptoids by introducing more nonpolar side chains such as longer alkyl or fluorinated side chains. (56-59)

### Passivation and Preferential Attachment of Biomolecules

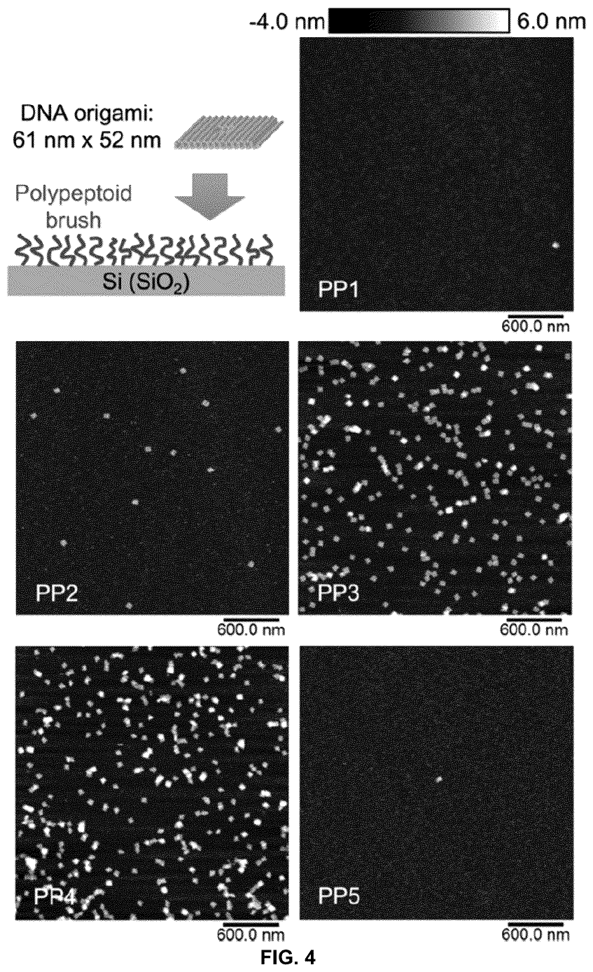

Tunable affinity toward biomolecules is a highly desired property of surfaces. Here, we demonstrate that polypeptoid brush monolayers are capable of tuning the affinity of surfaces toward DNA origami nanostructures (61×52×8 nm cuboid nanostructure with an aperture). Among the five polypeptoids, two polypeptoids are identified as good surface passivation molecules with either methoxyethyl or butyl side chains, while polypeptoids containing aromatic groups induce preferential attachment of DNA origami nanostructures on corresponding surfaces (FIG. 4). Previous strategies for surface passivation against DNA origami are commonly achieved with a hydrophobic trimethylsilyl layer produced by hexamethyldisilane (IMDS). (60-63) The binding of DNA origami on surfaces is most commonly mediated through electrostatic interactions, using Mg²⁺ as the electrostatic bridge between negatively charged DNA origami and negatively charged surfaces (e.g., Si, mica). (60,64) Here, we discovered that the binding affinity of DNA origami is decoupled from the surface hydrophilicity. Both PP1 and PP5 modified surfaces exhibit minimal affinity toward DNA origami, while these two surfaces are the most and the least hydrophilic (water contact angle: 38.9±0.7° vs 69.2±0.1°) among the five polypeptoid brush monolayer modified surfaces. PP3 and PP4 with a high aromatic monomer composition (75% and 100%, respectively) enable preferential attachment of DNA origami on surfaces, with a much higher density compared to the commonly used activated Si substrates under the same deposition conditions (FIG. 20). The results indicate the possibility of aromatic groups driving DNA origami attachment on surfaces, yet in this study, we do not attempt to further elucidate the underlying molecular mechanism.

### Specific Binding of Biomolecules

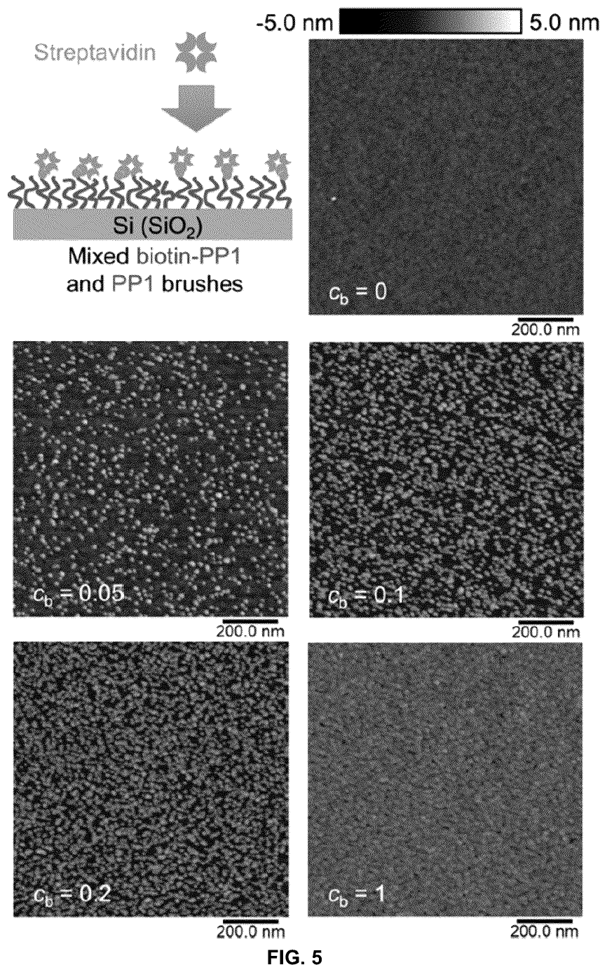

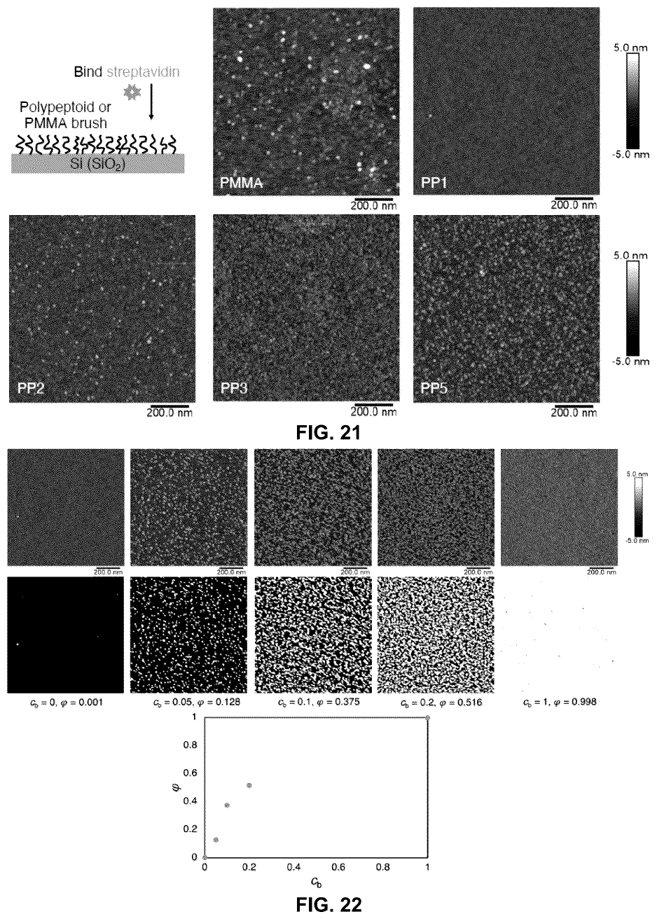

Specific protein binding events typically involve biomolecular recognition between specific chemical groups and the protein. Here, we show that as a surface modification material, functionalized polypeptoid brushes enable specific binding of proteins with a demonstration based on biotin-streptavidin interactions. PP1 is first identified to generate modified surfaces with minimal nonspecific binding of streptavidin (FIG. 21). This is within expectation as polypeptoid brushes with methoxyethyl side chains have been reported in multiple studies for creating antifouling surfaces against lysozyme, fibrinogen and serum proteins, fibroblast cells, and bacteria. (65-67) Next, biotinylated PP1 is synthesized by biotinylation of the N-terminus as the last step during solid-phase synthesis (FIG. 24, panel a). Therefore, the immobilization of streptavidin on the biotin-PP1 modified surface is enabled by the specific biotin-streptavidin interactions, where nonspecific adsorption of streptavidin is minimized. As shown in FIG. 5, different streptavidin binding density on surfaces is achieved through tuning the relative concentration of biotin-PP1 in PP1, c_{b}, and then grafting the polypeptoid mixtures onto Si substrates (streptavidin fractional surface coverage, φ, is quantified and plotted as a function of c_{b} in FIG. 22.

### Nanoscale Surface Patterns of Polypeptoid Brushes

### Compatibility of Polypeptoid Brushes with Lithographic Nanopatterning

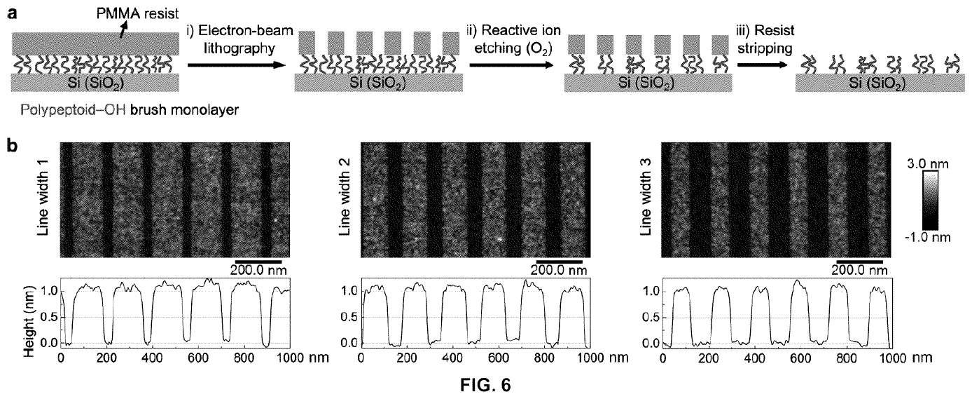

The compatibility with lithographic patterning of the polypeptoid brush monolayers is an important attribute of this class of bioinspired, sequence-defined polymers as surface modification materials as it will enable the generation of chemical contrast nanopatterns by design with monomer-level control. The lithographic patterning workflow adopted in this study follows a typical strategy of patterning surface modification layers on Si substrates. (52,61,68,69) As shown in FIG. 6 (panel a), it involves the following steps: (i) spin coating an ˜40 nm PMIMA resist on top of the polypeptoid brush monolayer for electron-beam lithography, with a subsequent development step; (ii) reactive ion etching with oxygen plasma to transfer the pattern of the PMMA resist layer into the underlying polypeptoid brush monolayer; (iii) stripping the PMMA resist and reannealing the polypeptoid brush to obtain a nanopatterned polypeptoid brush monolayer on Si substrates. AFM height profiles show that the generated line-space patterns are well-defined with sharp edges and reveal that the polypeptoid brush monolayers are ˜1 nm in thickness (FIG. 6, panel b). With the monolayer thickness obtained from nanopatterned polypeptoid brushes, the estimated grafting density (σ) based on the formula

$$σ = \frac{ρ {N}_{A} d}{{M}_{w}}$$

is ˜0.24 chain nm⁻², where ρ is the polypeptoid density (taken as 1.2 g cm⁻³ based on reported values in the literature), (70-72) N_{A} is Avogadro's number, d is the brush thickness, and M_{w} is the molecular weight of polypeptoids (here, the nanopatterned polypeptoid brush is PP3 with a M_{w}, of 2971 g mol⁻¹). As aforementioned, these polypeptoid 21-mers should have a radius of gyration (R_{g}) of ˜1 nm and a contour length of ˜7 nm based on previous SANS measurements of similar polypeptoids in close to θ-conditions. (41,42) With the brush thickness comparable to R_{g}, it suggests that the polypeptoid brush grafting density is relatively low, possibly in a “mushroom” regime close to an overlap density, as the brush top surface still appears smooth. Using the “mushroom” to “brush” transition (Σ=σπR_{g}²˜1), (39,73,74) it suggests the polypeptoid grafting density is near or below this transition σ˜0.26 chain nm⁻², which is consistent with the earlier estimation. A relatively low grafting density is common in brushes prepared via the “grafting to” method. (24,40)

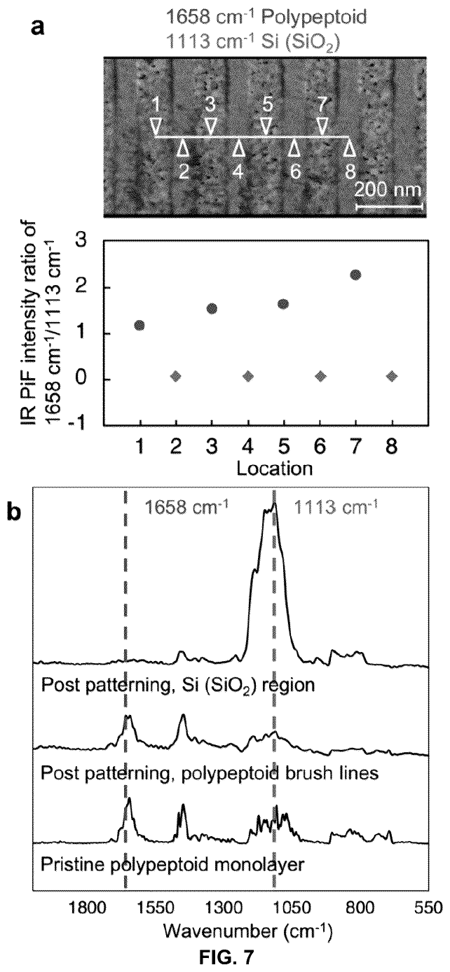

To further probe and confirm the chemical characteristics of polypeptoid brush monolayers post-lithographic patterning, the nanopatterned brush monolayers are mapped with IR PiFM at two wavenumbers, 1658 and 1113 cm⁻¹, with the former corresponding to the amide C═O stretching of polypeptoids and the latter corresponding to the Si—O—Si stretching of the native oxide layer at the Si substrate surface. The combined PiFM image shows the line-space patterns are well-defined chemically, with the Si (SiO₂) trenches with unobservable polypeptoid residues, as indicated by the near-zero PiF-IR signal intensity ratio of 1658 cm⁻¹/1113 cm⁻¹ at locations 2, 4, 6, and 8 (FIG. 7, panel a). More importantly, comparing the averaged full PiF-IR spectrum of sampled locations on the nanopatterned polypeptoid brush monolayer (locations 1, 3, 5, and 7) and the spectrum of a pristine polypeptoid monolayer before lithographic patterning, no distinct difference is observed between the two PiF-IR spectra, with the characteristic peak of polypeptoid amide C═O stretching at ˜1660 cm⁻¹ clearly observed post-electron-beam lithographic patterning (FIG. 7, panel b). This evidence indicates that the chemical characteristics of this polypeptoid brush-based system are preserved through the lithographic workflow which involves harsh conditions including background electron-beam radiation, oxygen plasma etching, and various organic solvents used in the process.

### Generation of Chemical Contrast Nanopatterns Consisting of Two Alternating Polymer Brushes

Generation of robust, well-defined chemical contrast patterns at desired resolution is of critical importance for applications such as biological assays for sensing and diagnostics, (11,75) surfaces for cell adhesion and growth, (76,77) directed self-assembly of block copolymers, (52,68,69,78) and site-specific immobilization of nanoscale objects such as DNA origami nanostructures and gold nanoparticles. (19,60-62) In particular, leveraging different polymer brushes to modify the corresponding lithographically defined regions is a common strategy to generate chemical contrast nanopatterns, where the effects from potential brush interpenetration could be minimized by molecular weight engineering per specific application requirements. (50-52) With a sequence-defined polymer brush platform, the parameter space of chemical contrast nanopatterns can be further expanded with precise, monomer-level engineering of the chemical functionalities.

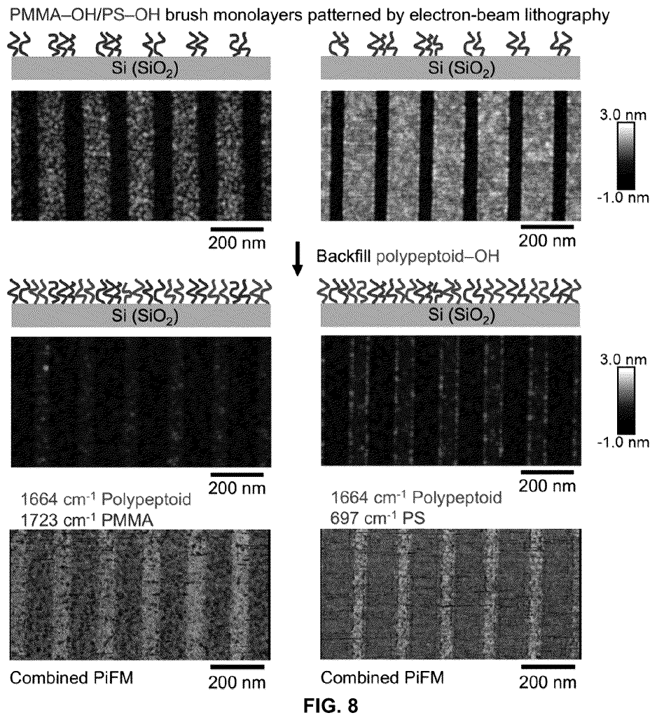

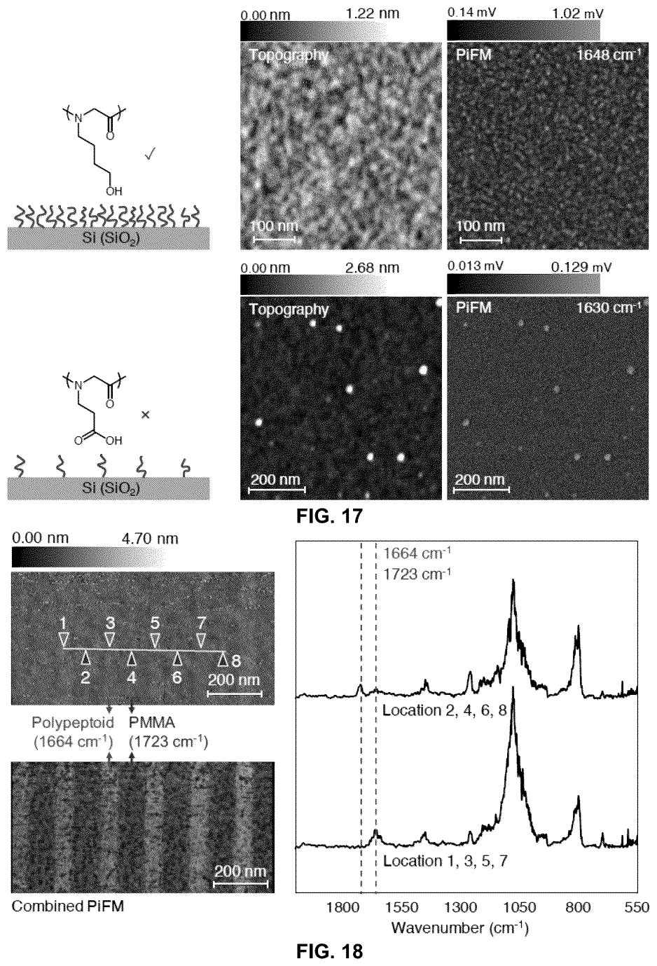

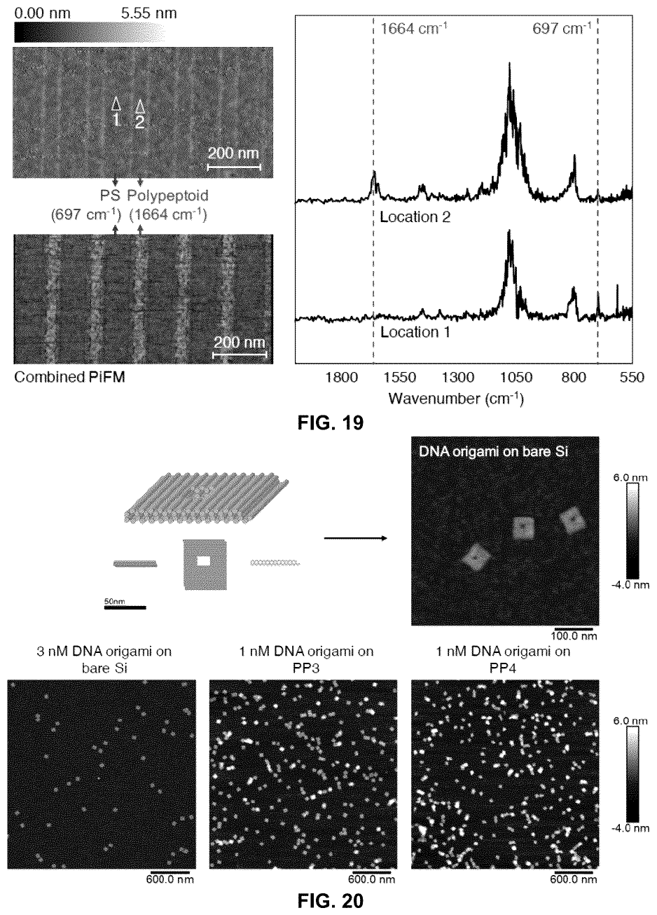

To generate chemical contrast nanopatterns with —OH terminal functionalized polymers, we demonstrate a workflow by nanopatterning a first polymer brush monolayer grafted on a Si substrate, followed by backfilling a second —OH functionalized polymer brush to graft onto the exposed Si (SiO₂) trenches, to form chemical contrast nanopatterns that consist of two polymer brushes. As shown in FIG. 8, a PS-OH or PMMA-OH brush monolayer is first nanopatterned by electron-beam lithography to generate PS-Si (SiO₂) or PMMA-Si (SiO₂) line-space nanopatterns, followed by backfilling a polypeptoid-OH brush. The successful grafting of —OH functionalized polypeptoids is evidenced by the topographical changes observed with AFM after the polypeptoid-OH backfill step, where the Si (SiO₂) trenches are grafted with polypeptoid brushes, resulting in almost coplanar PS-polypeptoid or PMMA-polypeptoid nanopatterns. The chemical characteristics of the generated nanopatterns of alternating PS (or PMMA) and polypeptoid brush line patterns are further confirmed by IR PiFM mapping at the characteristic bands for polypeptoid (1664 cm⁻¹), PMMA (1723 cm⁻¹), and PS (697 cm⁻¹), respectively (FIG. 8). Notably, while both polypeptoid and PMMA have carbonyl groups, the C═O stretching bands of the two polymers show up ˜60 cm⁻¹ apart (peak at 1664 cm⁻¹ for amide C═O stretching in polypeptoid, peak at 1723 cm⁻¹ for ester C═O stretching in PMMA) in the full PiF-IR spectra collected between 2000 and 541 cm⁻¹ (FIG. 18), well within the tool resolution limit to clearly distinguish between the grafted PMMA and polypeptoid brushes of ˜1 nm thickness. While the goal here is not to demonstrate the highest resolution possible to pattern polypeptoid brush monolayers, the successful demonstration of nanopatterns with sub-100 nm features using electron-beam lithography, together with the versatile design possible with sequence-defined polypeptoids, makes this sequence-defined polypeptoid brush platform highly attractive for applications that require incorporation of specific chemical functionalities in the desired regions.

### Surface Chemical Contrast Nanopatterns for Selective Binding of Biomolecules

Biomolecular building blocks made of sequence-defined biomacromolecules (nucleic acids, proteins) are powerful programmable building blocks with nanometer resolution and addressability. (2) In nanobiotechnology, bioelectronic devices often require accurate placement of these biomolecular building blocks at desired locations for device functionality. (10,11,24) Here, we demonstrate the utility of these bioinspired, sequence-defined polypeptoid brushes as a highly versatile platform that enables precise and selective placement of biomolecular building blocks on nanopatterned surfaces.

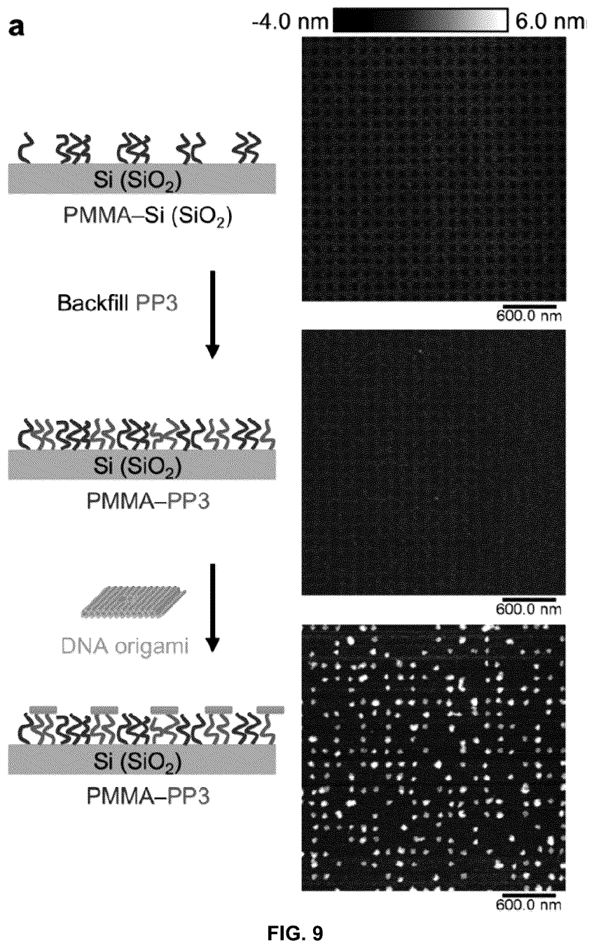

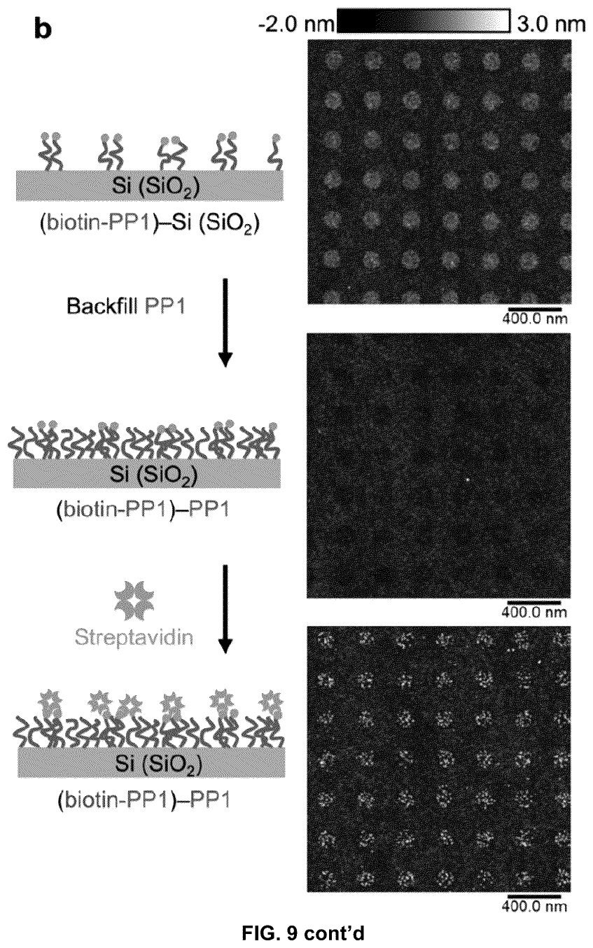

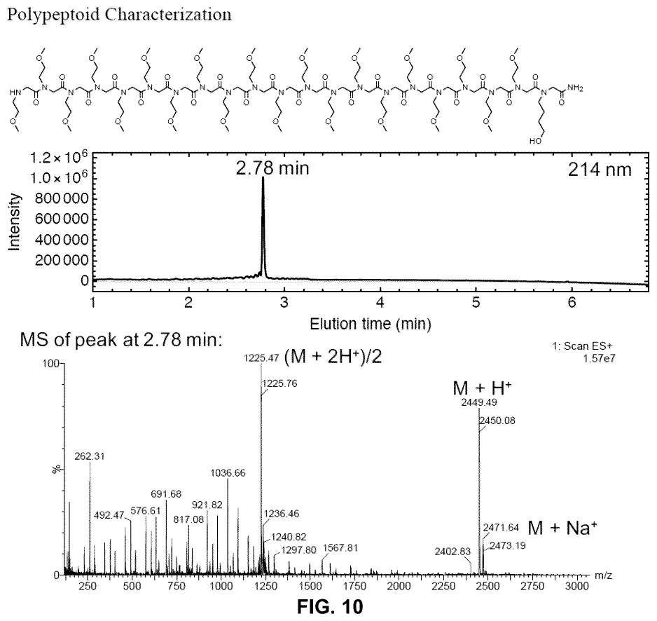

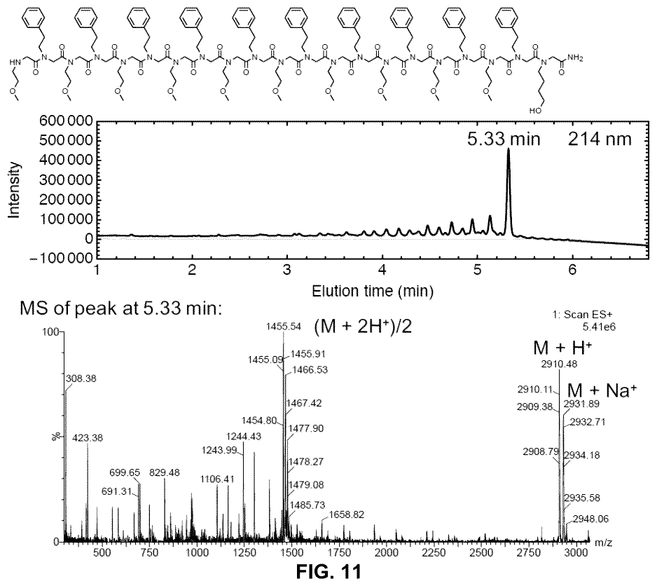

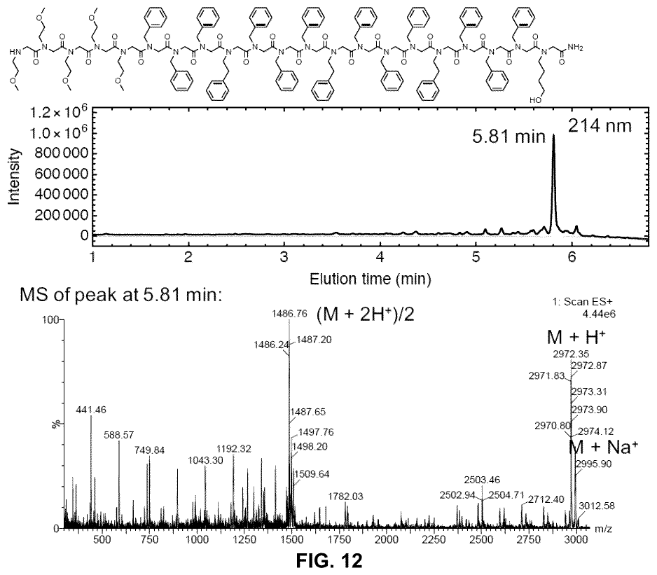

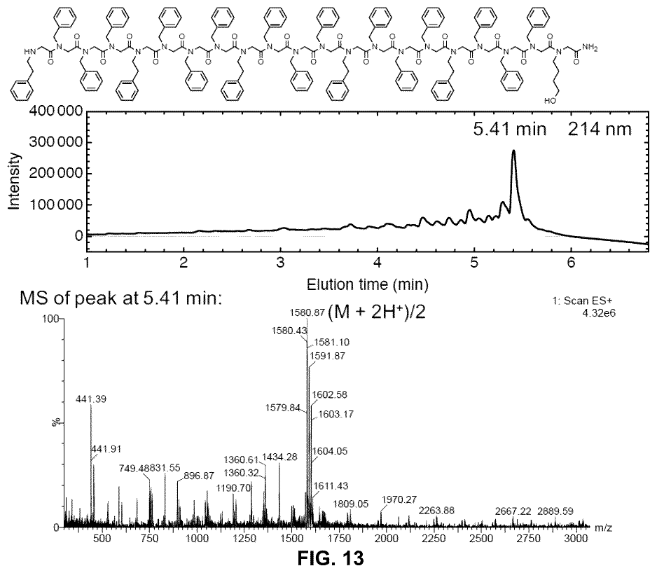

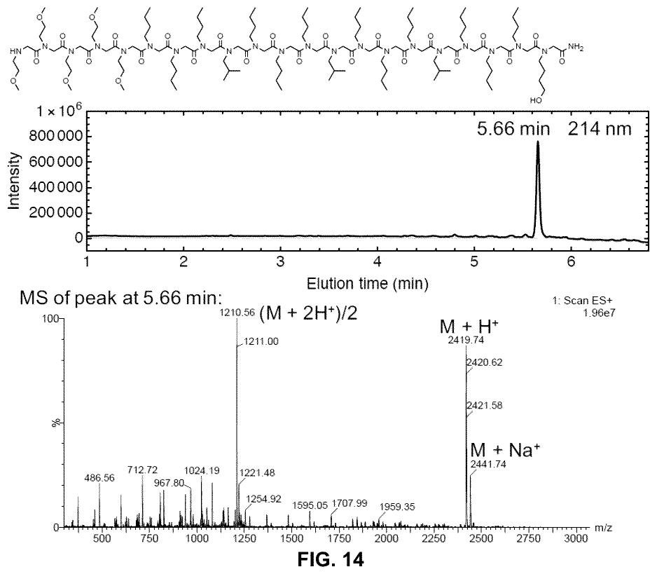

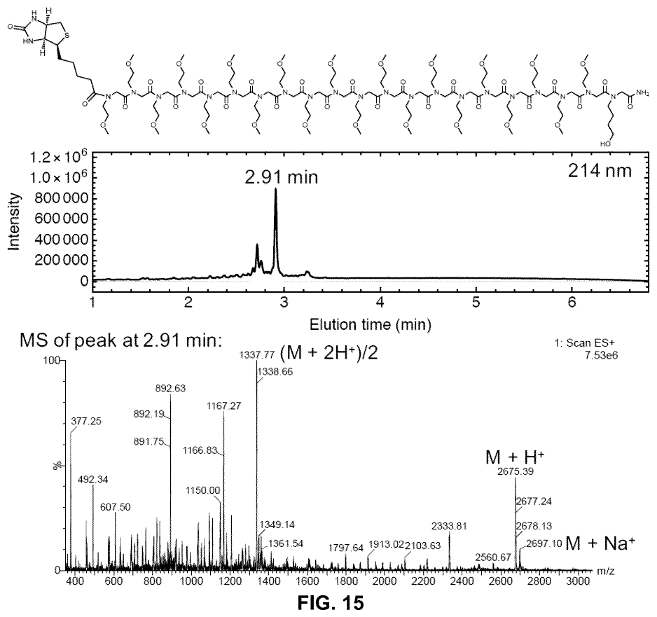

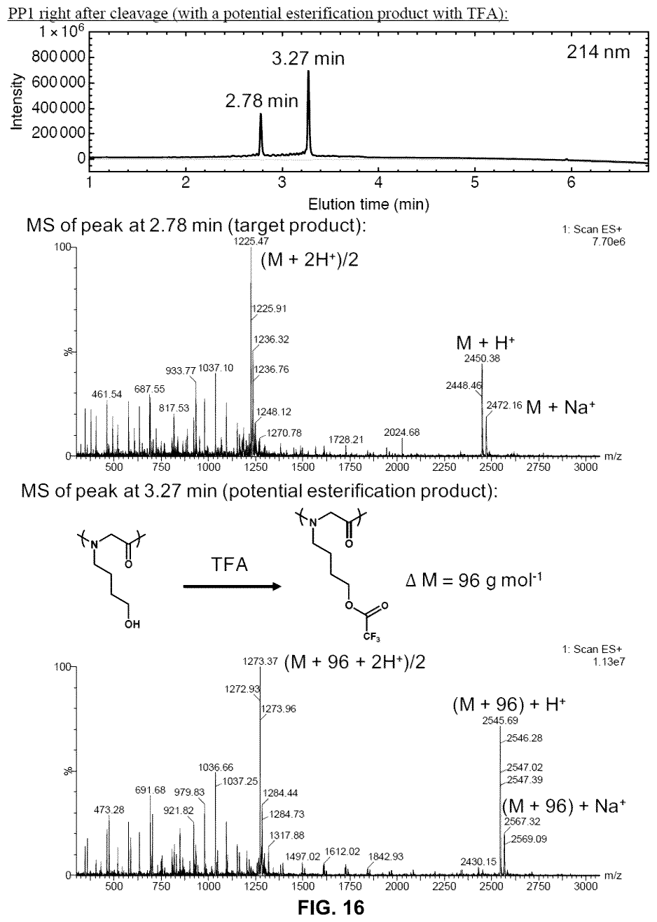

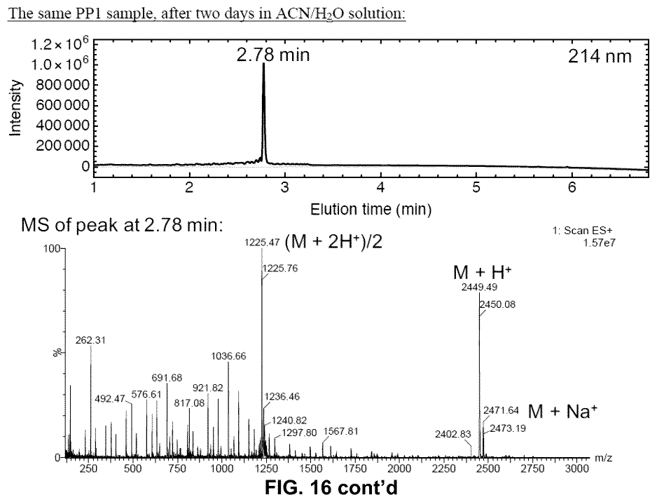

Previous strategies for selective placement of DNA origami (in some cases with controlled orientation) on lithographically patterned Si substrates leverage electrostatic interactions, with background passivation using a hydrophobic trimethylsilyl layer produced by hexamethyldisilane (HMDS). (60-63) Here, using the identified polypeptoid brush (PP3) with high DNA origami binding affinity and a PIMMA brush as the surface passivation layer for the background, surface chemical contrast nanopatterns with polypeptoid-modified circular patches of commensurate feature size are fabricated for selective binding of the 61 nm×52 nm DNA origami nanostructures (FIG. 9, panel a). Previous studies on individual DNA origami placement and orientation on lithographically patterned surfaces have reported stringent deposition and rinsing conditions. (61,62) Here, using the PMMA-PP3 chemical contrast nanopattern, we can achieve a relatively high binding site occupancy of individual DNA origami without complicated deposition and rinsing protocols. While achieving high individual DNA origami occupancy is not the focus of this study, we believe this polymer brush-based strategy, with robust background passivation and high yet tunable affinity rendered by sequence-defined polypeptoids, will expand the tool box for precise placement and assembly of DNA origami on nanopatterned surfaces, potentially as a more process-tolerant strategy.

Generation of well-defined, surface immobilized protein nanoarrays is important for immunoassays and pharmaceutical screening applications, as well as for proteomics research. (75,79) Here, following a similar strategy in designing surface chemical contrast nanopatterns for selective binding, a good passivation background against streptavidin is achieved with a PP1 brush monolayer that has minimal nonspecific binding of streptavidin, and surface regions for specific binding of streptavidin are modified by biotin-PP1 brushes. This strategy successfully yields surface chemical contrast nanopatterns (biotin-PP1-PP1) with excellent selective immobilization of streptavidin on circular patches at length scales defined by electron-beam lithography (FIG. 9, panel b). In this case, both the background and the binding regions have minimal nonspecific binding of streptavidin, and the immobilization of streptavidin on the nanopattern is only mediated via the specific biotin-streptavidin interactions.

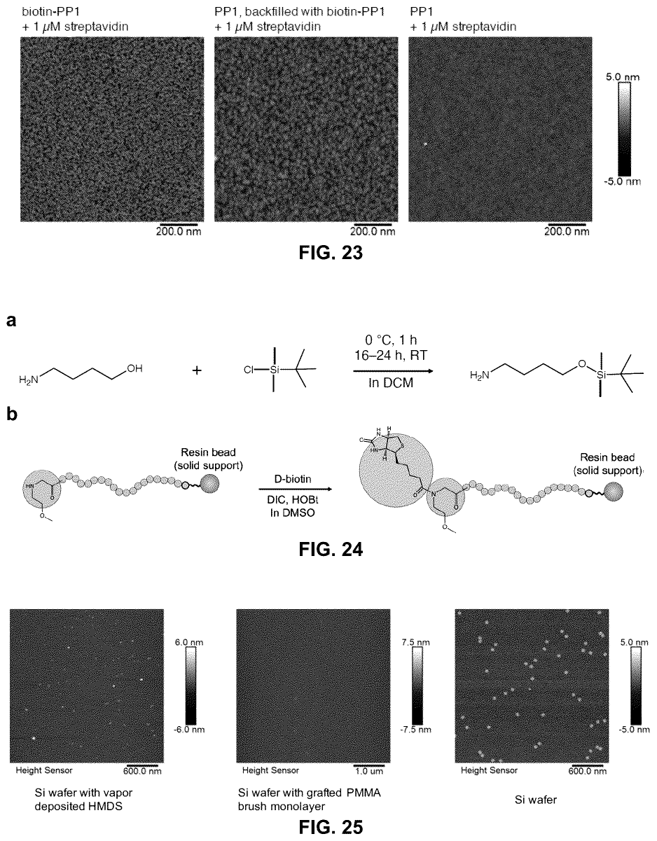

In this chemical contrast nanopattern design for selective immobilization of streptavidin, effects from potential insertion of the second polymer brush into the nanopatterned first polymer brush monolayer during the backfill step need to be mitigated. It is found to be necessary that the biotinylated polypeptoid brush monolayer is lithographically patterned first, and the nonbiotinylated polypeptoid brush for background passivation is backfilled as the second polymer brush; otherwise, the interdigitated biotinylated polypeptoid brush will bind a sufficient amount of streptavidin, making the background nonpassivating against streptavidin (FIG. 23). This adjusted patterning strategy indicates that the covalently attached biotin functionality on polypeptoid brushes is preserved through the lithographic patterning workflow, enabling specific biotin-streptavidin binding in the final surface chemical contrast nanopatterns.

### CONCLUSIONS

In summary, we have introduced a class of surface modification materials based on bioinspired, end-grafted polypeptoid brushes. The sequence-defined synthesis of these polypeptoid brushes enables precise customization of the polypeptoid composition and sequence to match the desired functionality, processability, and biocompatibility. This material platform offers a wide spectrum of possibilities for tailoring molecular interactions at the inorganic/bio interface, ranging from simple surface energy modification (hydrophilic/hydrophobic) to various degrees of antifouling/preferential attachment properties and specific biomolecular recognition. The “grafting to” approach enhances versatility across different substrates, while compatibility with nanoscale lithographic patterning provides opportunities for engineering semiconductor/bio interfaces with single-molecule-level addressability. We demonstrated uniform, ultrathin (˜1 nm) surface modification layers capable of mediating diverse interfacial interactions through a range of polypeptoid composition and sequences. These polypeptoid brush monolayers were used to generate highly precise chemical contrast nanopatterns defined by electron-beam lithography. Importantly, we verified the preservation of chemical functionalities of the polypeptoid brushes post-lithographic patterning. By selecting appropriate polypeptoid brushes for surface passivation or target biomolecule binding, we achieved the selective immobilization of DNA origami nanostructures and streptavidin on these nanopatterns.

We believe this bioinspired, sequence-defined polypeptoid brush platform will be of interest as surface modification materials beyond the current scope of this study aimed at semiconductor/bio interfaces. The thermal stability, (32) enzymatic resistance, (33-35) and control over solubility make polypeptoids advantageous over natural biomacromolecules like polypeptides and nucleotides for more tolerant processing conditions, while sequence-definition renders polypeptoids the same level of programmability and precision in molecular design as biomacromolecules and potentially more flexibility in incorporation of chemical functionalities. We expect this polypeptoid brush platform could find immediate applicability in biophysical research and nanobiotechnology applications that utilize nanopatterned surfaces and structures such as for cell adhesion and signaling (77,80) and semiconductor-biomolecule hybrid sensing systems, (4) as well as peptide/protein sequencing. (81,82)

### METHODS

### Materials

Solvents and reagents were purchased from commercial suppliers and used without further purification. Hydroxyl functionalized polystyrene (2700 g mol-1) and poly(methyl methacrylate) (6300 g mol-1) were purchased from Polymer Source (Dorval, Canada). Si substrates (prime grade, with a native oxide layer) were sourced from Addison Engineering Inc. (San Jose, CA, United States). DNA origami nanostructures (Prefabricated nanostructure PF-2, 61×52×8 nm cuboid with a 9×15 nm aperture, honeycomb lattice) were sourced from Tilibit Nanosystems (Munich, Germany).

### Solid-Phase Synthesis of Polypeptoids

Polypeptoids were synthesized on a custom robotic synthesizer using rink amide resin (100-200 mesh, Novabiochem) with intermediate loading (˜0.64 mmol g⁻¹) and commercially available submonomers, following reported procedures. (83) The submonomer 4-amino-1-butanol for introducing the hydroxyl group was protected by tert-butyldimethylsilyl (tBDMS) (FIG. 24, panel a) when it was used for solid-phase synthesis. Rink amide resin (50 mol) was first swelled in N,N-dimethylformamide (DMF) for 10 min and deprotected with 4-methylpiperidine (1 mL, 20% v/v in DMF). Bromoacylation was performed by adding bromoacetic acid (1 mL, 0.8 M in DMF) and N,N′-diisopropylcarbodiimide (DIC) (1 mL, 0.8 M in DMF) and mixing for 20 min. Nucleophilic displacement was performed by adding the corresponding amine submonomers (1 mL, 1 M in DMF) and mixing for 1 h. The resin was washed with DMF after each synthetic step. At the end of the synthesis, the resin was washed with DMF and then with dichloromethane (DCM) and dried with a nitrogen flow.

To synthesize polypeptoids with a biotin group, biotinylation was performed on the N-terminus of polypeptoids (on resin) by coupling with D-biotin in dimethyl sulfoxide (DMSO) (FIG. 24, panel b). 16 equiv of D-biotin (0.4 M in DMSO), 16 equiv of hydroxybenzotriazole (HOBt), and 16 equiv of DIC (0.4 M in DMSO) were added to swelled resin and mixed overnight, followed by washes and drying as noted above.

Polypeptoids were cleaved from the resin using a trifluoroacetic acid (TFA) cocktail (95% TFA, 5% H₂O) for 1 h. The typical cleavage scale is 25 mol of resin in 3 mL of the TFA cocktail. The resin was filtered and rinsed with another 2 mL of cleavage cocktail and then rinsed with 5 mL of DCM three times. The collected solutions were dried in vacuo on a Biotage V10 and lyophilized from acetonitrile (ACN):H₂O (1:1, v/v) solutions to yield the final product.

The design of polypeptoids takes into consideration crystallization inhibition. In PP3 and PP4 that have a long aromatic block, a monomer with phenylethyl side chain (Npe) is placed between every three monomers with phenylmethyl side chain (Npm), where the Npe monomer with one —CH₂— longer linker serves as a crystallinity disruptor, as blocks of aromatic monomers with uniform linker length are highly crystalline and insoluble. (56) Similarly, in PP5, monomers with isobutyl side chain (Nib) are used to disrupt the otherwise crystalline block of monomers with uniform n-butyl side chains (Nbu). (56)

### Characterization of Polypeptoids with Ultrahigh-Pressure Liquid Chromatography Mass Spectrometry (UPLC-MS)

UPLC-MS was performed on a Waters Xevo G2-XS, equipped with a time-of-flight mass spectrometer. Polypeptoid samples were dissolved at ˜0.5 mg mL⁻¹ in ACN:H₂O (1:1, v/v) and run at an eluent gradient from 5% ACN/95% H₂O to 95% ACN/5% H₂O (with 0.1% TFA) over 6.8 min on a C18 or C4 column.

### Preparation of Polymer Brush Monolayers Grafted on Si Substrates

Lyophilized polypeptoids with an —OH group were dissolved in dichloroethane (DCE) at 0.5 wt %, and PS-OH and PMMA-OH powders were dissolved in toluene at 1.5 wt %. Polymer solutions were filtered through 0.45, 0.2, and 0.02 m PTFE filters. Si substrates were pretreated with UV-ozone for 5 min, and then, polymer solutions were spin coated at either 2000 rpm (polypeptoids) or 3000 rpm (PS-OH, PMMA-OH). The spin coated polypeptoid thin films were annealed at 180° C. for 30 min, and PS-OH and PMMA-OH thin films were annealed at 200° C. for 30 min, under vacuum with 10 sccm N₂ flow. The thin films on Si substrates were then sonicated in N-methyl-2-pyrolidone (NMP) for 5 min, 3 times, to remove the excess ungrafted polymer chains, followed by sonication in isopropyl alcohol (IPA) for 5 min to remove the high-boiling NMP solvent. The rinsed substrates were annealed at 100° C. for 10 min under vacuum with 10 sccm N₂ flow to remove residue solvents and leave a relaxed polymer brush monolayer grafted on the Si substrate.

### Characterization of Polymer Thin Films and Brush Monolayers on Si Substrates

Ellipsometry measurements were performed on a JA Woollam M-20000 DI ellipsometer using a Cauchy model for the polymer layer. Infrared spectroscopy measurements were performed on a Thermo-Fisher Nicolet iS50 FTIR equipped with a variable angle reflectance accessory by Harrick VariGATR and a germanium crystal. X-ray photoelectron spectroscopy (XPS) measurements were taken on a Thermo-Fisher K-Alpha Plus XPS/UPS instrument with a monochromatic Al X-ray Source (1.486 eV). Survey spectra (1350 to −10 eV) were taken with 1 eV steps; high resolution O 1s, N 1s, C 1s, and Si 2p spectra were taken with 0.1 eV steps.

### Water Contact Angle and Surface Free Energy Measurements

Measurements were performed on a KRUSS DSA 100E tool. Surface free energy was measured and calculated using the “two-liquid geometric approach” (84-86) with water as the polar solvent and diiodomethane as the nonpolar solvent. Static liquid contact angles are measured by capturing an image immediately after dispensing a 2 μL liquid drop on the substrate. For each substrate, 3-5 different locations were measured for liquid contact angle, with one measurement at each location.

### Electron-Beam Lithography and Generation of Surface Nanopatterns

PMMA (950 000 g/mol, 1% in chlorobenzene) was used as a positive-tone electron-beam resist. Resist was spin coated onto a Si substrate grafted with a polymer brush monolayer (polypeptoid-OH, PS-OH, or PMMA-OH) at 3000 rpm to give a thickness of ˜40 nm and then baked at 180° C. for 5 min. The line-space or circular patterns were exposed on a Raith EBPG5200 ultra high-performance electron beam lithography system at 100 kV and 2 nA beam current, with dose ranges optimized for different nanopattern designs (dose range: 1500-3000 μC cm⁻², 700-1000 μC cm⁻²). The resist was then developed using a high contrast cold development process by sonicating in IPA/water (7:3, v/v) at 5° C. for 100 s. The exposed polymer brushes were then dry etched using oxygen plasma on an Oxford PlasmaLab 150 Inductively Coupled Etcher (ICP) with 70 W HF power, 130 W ICP power at 4 mTorr, 20° C., with gas flows of O₂ (30 sccm) and He (50 sccm). The remaining resist was then stripped by sonication in NMP for 5 min, 3 times, followed by sonication in IPA for 5 min to remove the high-boiling NMP solvent. The rinsed substrates were annealed at 100° C. for 10 min under vacuum with 10 sccm N₂ flow to remove residue solvents and leave a relaxed polymer brush monolayer grafted on the Si substrate. For surface nanopatterns that consist of two polymer brushes, the second polymer brush was backfilled to the exposed Si(SiO₂) surface by spin coating, annealing, rinsing, and reannealing, following the same procedure as preparing the first polymer brush monolayer that was patterned (note: the backfill of the second polymer brush was performed right after the resist stripping step to minimize deactivation of the silanol groups on the just exposed Si substrates).

### AFM

Atomic force microscopy (AFM) measurements were taken on a Bruker Dimension Icon AFM, with either a noncontact tapping mode or PeakForce tapping mode.

### IR PiFM

Infrared photoinduced force microscopy (IR PiFM) measurements were conducted at Molecular Vista Inc. on a Vista One microscope, with a PT277-XIR laser from Ekspla (Vilnius, Lithuaia) as the excitation source with a full tuning wavenumber range between 7143 and 541 cm⁻¹ and a spectral line width of ˜3 cm⁻¹. For fixed wavenumber PiFM imaging, PiF-IR spectra were first taken on the polymer brush monolayers and substrates (patterned or nonpatterned); then, the IR wavenumber with peak intensity in the corresponding IR range of the characteristic chemical functionality of the polymer/substrate was picked for PiFM imaging (polypeptoids: amide C═O stretching ˜1660 cm⁻¹; PMMA: ester C═O stretching ˜1720 cm⁻¹; PS: aromatic C—H bending ˜700 cm⁻¹; Si substrates with a native oxide layer: Si—O—Si stretching ˜1110 cm⁻¹). All images were collected with a scan speed of 0.5 Hz. PiF-IR spectra were power normalized and acquired with a sweep time of ˜13 s. Platinum-iridium-coated NCH 300 kHz noncontact cantilevers from Nanosenors (Neuchatel, Switzerland) were used for all measurements. Surface Works was used for all of the image and data processing.

### Selective DNA Origami, Streptavidin Immobilization on Surface Nanopatterns

Upon generation of the corresponding surface nanopatterns, a 150-200 μL drop (sufficiently large to cover the entire nanopatterned area) of DNA origami solution (1 nM in Tris buffer, 40 mM MgCl₂, pH=8.5-9) or streptavidin solution (100 nM in 1×PBS buffer) was deposited on the nanopatterned substrate. The solution drop was then incubated on the substrate in a moisturized chamber for 1 h, followed by the corresponding rinsing protocols and drying with a N₂ stream. Rinsing protocol: DNA origami incubated samples, immerse the substrate in 20-30 mL of deionized water for 2 min, twice; streptavidin incubated samples, immerse the substrate in 20-30 mL of 1×PBS buffer for 2 min, twice. The same incubation protocol of DNA origami or streptavidin (with concentration noted in corresponding samples) on nonpatterned substrates modified with polymer brush monolayers was adopted. For nonpassivating surfaces against streptavidin, an additional deionized water rinse was used, as salt deposits from the PBS buffer occur after large amounts of streptavidin adsorb on the incubated surface area.

### Additional Information on Material Synthesis

4-[(tert-butyldimethylsilyl)oxy]butan-1-amine. Tert-butyldimethylsilyl (tBDMS) protection of 4-amino-1-butanol was carried out following a similar protocol reported previously.1 A solution of tert-butyldimethylsilyl chloride (tBDMS-Cl, 2.1 g, 14 mmol) in dichloromethane (DCM, 2.6 mL) was added dropwise at 0° C. to a vigorously stirred solution of 4-amino-1-butanol (5 g, 56 mmol) in DCM (1.3 mL), and stirring was continued for 1 h at 0° C. The mixture was allowed to warm to room temperature and stirred for an additional 16-24 h. The crude mixture was poured into water (20 mL), and the layers were separated. The organic layer was washed with water (2×20 mL), and the aqueous layers was re-extracted with DCM (2×20 mL). The combined organic extracts were dried over Na2SO4, then the solvent was removed in vacuo, yielding 2.69 g (94%) product of a colorless oil. See FIG. 24 (panel a).

Biotinylation of polypeptoids. Rink amide resin with polypeptoid (25 μmol) was re-swelled in N,N-dimethylformamide (DMF) for 20-30 min, drained. D-biotin (0.4 M, 16 equiv.) and hydroxybenzotriazole hydrate (HOBt·xH2O, 0.4 M, 16 equiv.) in dimethyl sulfoxide (DMSO, 1 mL) were added, followed by diisopropylcarbodiimide (DIC) (0.4 M, 16 equiv.) in DMSO. The syringe reaction vessel was allowed to mix on a rocker for 20-24 h. The resin was washed with DMF, then dichloromethane (DCM), and dried with a nitrogen flow. See FIG. 24 (panel b).

TABLE 1
Comparison of brush monolayer thickness and water contact
angle of the correspondingly modified Si substrate surface
between —OH and —COOH functionalized polymers.

| | | Monolayer | Water |
|---|---|---|---|
| | Mₙ (g mol⁻¹) | thickness (nm)^{a} | contact angle |
| PS-OH | 10,000 | 5.70 | (89.5 ± 0.1)° |
| PS-COOH | 10,000 | 0.48 | (56.0 ± 0.0)° |
| PMMA-OH | 6300 | 3.89 | (63.9 ± 0.6)° |
| PMMA-COOH | 8400 | <0 | (39.7 ± 3.9)° |

^{a}Polymer brush monolayers are prepared on Si wafers with a 250 nm thermal oxide layer, with the thermal oxide layer thickness measured via ellipsometry before polymer grafting.

TABLE 2
Surface free energy (SFE) of Si substrates modified
by different polypeptoid brush monolayers.

| | | Diiodo- | SFE (total) | SFE (disperse) | SFE (polar) |
|---|---|---|---|---|---|
| | Water | methane | (mN/m) | (mN/m) | (mN/m) |
| PP1 | (37.6 ± 0.3)° | (40.2 ± 0.6)° | 64.8 ± 0.5 | 39.5 ± 0.3 | 25.3 ± 0.2 |
| PP2 | (67.6 ± 0.4)° | (35.7 ± 0.4)° | 49.7 ± 0.4 | 41.7 ± 0.2 | 7.9 ± 0.2 |
| PP3 | (70.6 ± 0.1)° | (33.2 ± 0.1)° | 49.2 ± 0.1 | 42.9 ± 0.1 | 6.3 ± 0.1 |
| PP4 | (72.5 ± 0.1)° | (31.0 ± 0.2)° | 49.1 ± 0.1 | 43.8 ± 0.1 | 5.3 ± 0.1 |
| PP5 | (81.9 ± 0.5)° | (56.4 ± 0.3)° | 35.5 ± 0.4 | 30.6 ± 0.2 | 4.8 ± 0.2 |

Surface free energy is calculated using a “two-liquid geometric approach” ⁸⁴⁻⁸⁶ based on static contact angles of two liquids, water (polar) and diiodomethane (nonpolar), measured immediately after dispensing a 2 μL drop of the liquid with an automated dispenser.

We note here that the polypeptoids are soluble in water (PP1) or diiodomethane (PP2-PP5) to some degree. While measurements were possible on the (insoluble) grafted brush monolayers, it was not possible to verify the trend on thicker polypeptoid films. We also note that thicker films of polystyrene were observed to be soluble in diiodomethane, which made it not possible to measure and calculate surface free energy using the water-diiodomethane pair. However, we did not find acknowledgements of the difficulty of performing the measurements with the water diiodomethane when the polymers are soluble in the polar or nonpolar liquids. (84-86)

### Example 2

### Poly(methyl methacrylate) Brushes for Surface Passivation Against Nucleic Acid Nanostructures

FIG. 25. Comparison between using HMDS and PMMA brush for surface passivation of Si wafer substrates against DNA origami nanostructures (without surface passivation, a bare Si wafer binds to DNA origami nanostructures). Si wafer with vapor deposited HMDS does not have whole DNA origami (square shapes as seen on bare Si wafer), yet there are debris of materials on the surface after incubation of the DNA origami buffer solutions (it is also reported that the formed trimethylsilyl layer from HMDS undergoes hydrolyzation at basic pHs >9 or after long periods of soaking in incubation buffer1). Si wafer with grafted PMMA brush monolayer stays clean after incubation of DNA origami buffer solutions.

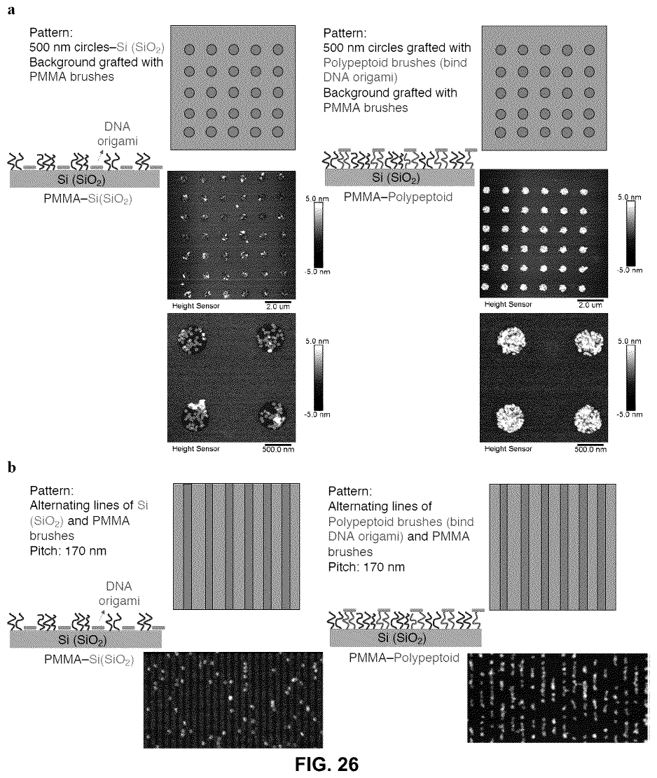

FIG. 26. Demonstration of selective binding of DNA origami nanostructures on lithographically patterned substrate surfaces, where the DNA origami nanostructures selectively bind to exposed Si (SiO2) surface, or polypeptoid brushes grafted surface, enabled by passivating the rest of the surface with PMMA brushes. (a) 500 nm circular features, (b) alternating line patterns with 170 nm pitch.

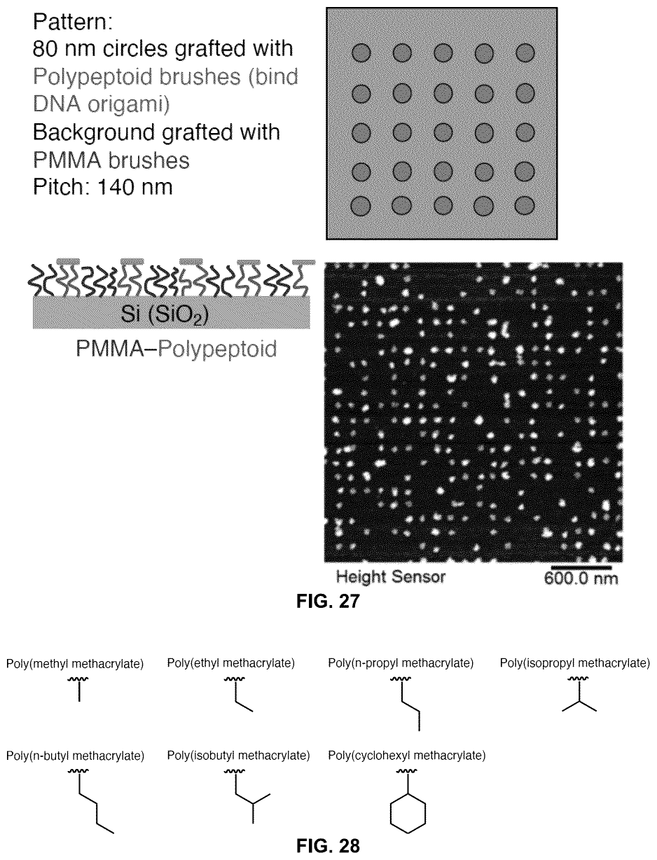

FIG. 27. A process of creating surface patterns that bind individual DNA origami nanostructures at designated locations. Lithographical features are created (on a background passivated with PMMA brushes) with dimensions comparable to individual DNA origami nanostructures, and make the features have preferential binding to DNA origami nanostructures to enable the selectivity.

FIG. 28. Exemplary R groups of the PMA.

### Example 3

### Polypeptoid Brush Monolayers for Selective Surface Passivation and Functionalization for Biomolecules

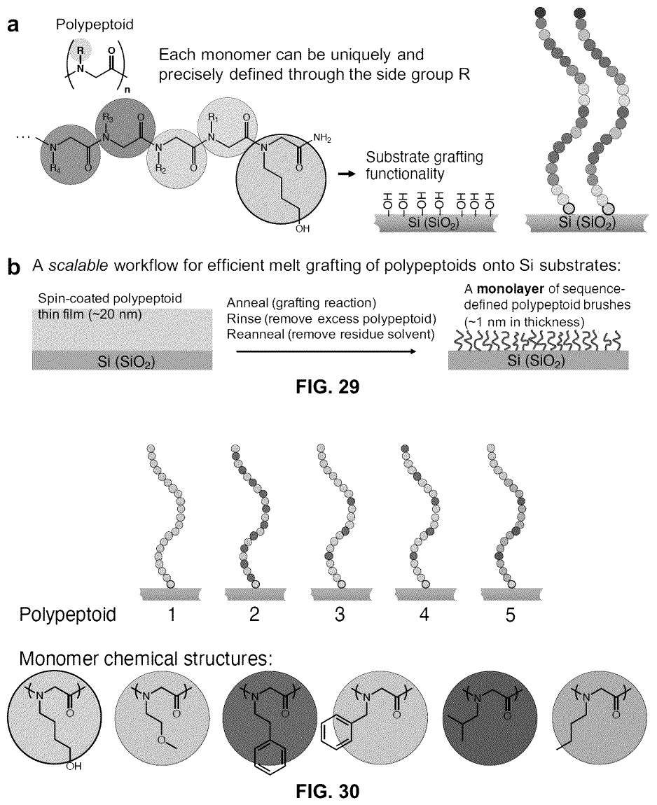

FIG. 29. Overview of the polypeptoid brush platform. a) Functionalized polypeptoids are synthesized as sequence-defined using solid phase synthesis. For the polypeptoids to covalently attach to the substrate surface (native SiO2 layer on Si wafer), here is introduced a hydroxy function on the first monomer as the side chain. b) The simple, robust, scalable workflow to prepare polypeptoid brush monomers on substrates.

FIG. 30. The chemical structures of the five polypeptoids described and used in the embodiments.

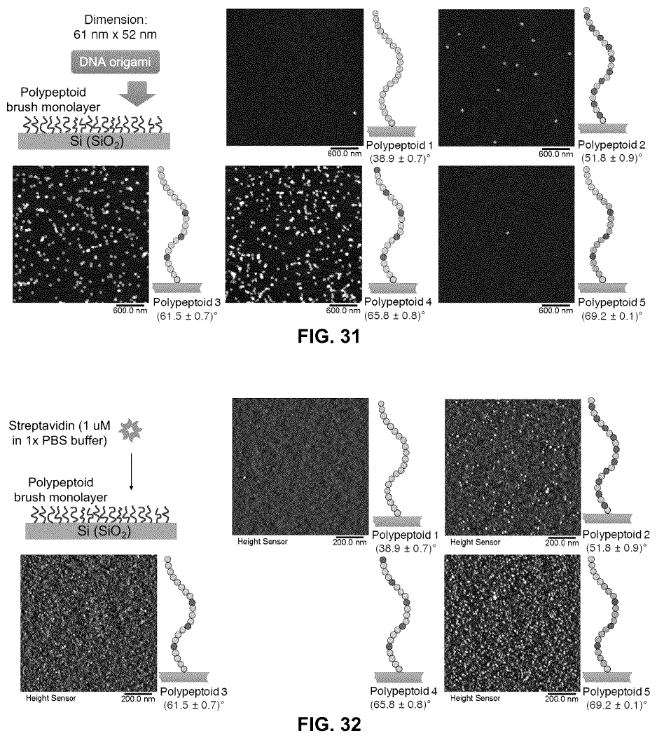

FIG. 31. With the five different polypeptoids, a monolayer of each polypeptoid brush is tethered to Si wafer substrates. The surface energy of the substrate is modified correspondingly with these about 1 nm thick polypeptoid brush monolayers, as indicated by the water contact angle in blue. The modified surfaces also display different binding affinity towards DNA origami, with Polypeptoid 1 and Polypeptoid 5 display minimal affinity to DNA origami, rendering them as excellent passivation surface modification layers against DNA origami, despite their distinct surface hydrophilicity. Polypeptoid 3 and Polypeptoid 4 are found to have high binding affinity towards DNA origami.

FIG. 32. Among the different polypeptoids, Polypeptoid 1 is identified as a good surface passivation material against streptavidin.

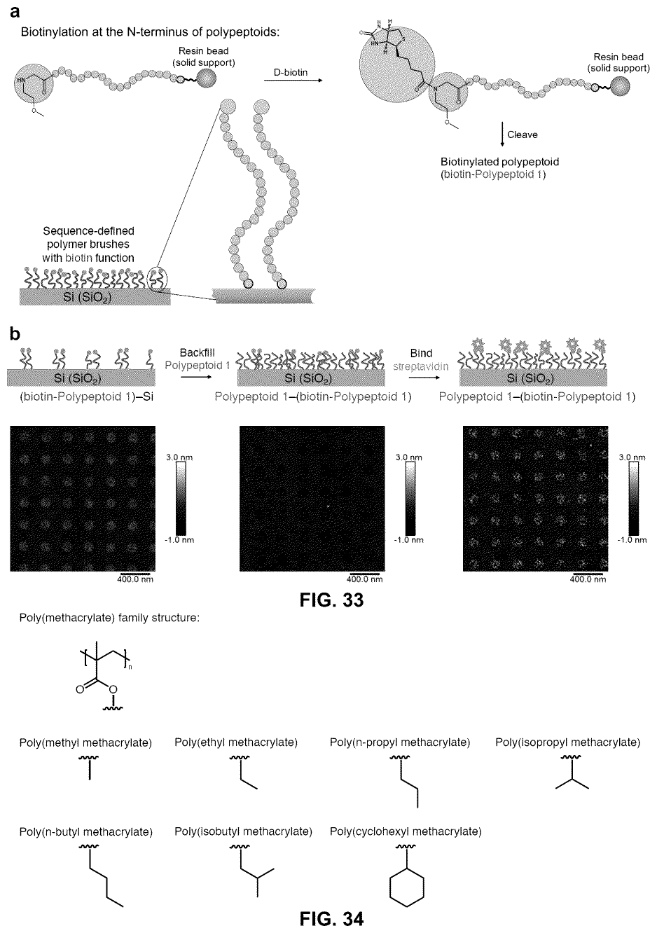

FIG. 33. a) Biotinylation of polypeptoids is achieved by reacting D-biotin with the free N-terminus of polypeptoid chains on resin as the last coupling step in the solid-phase synthesis. b) Surface nanopatterns with patches consisting of tethered biotin-Polypeptoid 1 brush for specific binding of streptavidin and background consisting of tethered Polypeptoid 1 for passivation again streptavidin is created and demonstrated to selectively bind to streptavidin via biotin-streptavidin interactions in the circular patches with diameters down to 100 nm.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A device comprising a substrate with a monolayer of a poly(methacrylate) (PMA) or polypeptoid polymer.

2. The device of claim 1, wherein the device comprising a substrate with a monolayer of a poly(methacrylate) (PMA).

3. The device of claim 1, wherein the monolayer of a poly(methacrylate) (PMA) is a poly(methyl methacrylate) (PMMA) tethered to a surface of the substrate to passivate the surface against adsorption of nucleic acid nanostructures.

4. The device of claim 3, wherein the PMMA brush is a linear polymer chain comprising a single functional group at one end where the functional group may be a hydroxyl (—OH), primary amine (—NH₂), thiol (—SH), or carboxylic acid (—COOH) functional group.

5. The device of claim 4, wherein the device comprises a patterned structure comprising a monolayer of functionalized PMMA polymer molecules tethered to selected patterned areas, producing alternating covered and non-covered regions of tethered PMMA brush molecules.

6. The device of claim 2, wherein non-passivated regions comprise a lateral dimension in the range of 10-1000 nm.

7. The device of claim 2, wherein the poly(methacrylate) (PMA) is a poly(methyl methacrylate), poly(ethyl methacrylate), poly(n-propyl methacrylate), poly(isopropyl methacrylate), poly(n-butyl methacrylate), poly(i-butyl methacrylate), or poly(cyclohexyl methacrylate).

8. The device of claim 1, wherein the device comprising a substrate with a monolayer of a polypeptoid polymer.

9. The device of claim 8, wherein the monolayer of functionalized polypeptoid (N-substituted polyglycines) molecules tethered to a substrate surface is used as a surface modification material for passivation or functionalization for binding to biomolecules.

10. The device of claim 9, wherein the polypeptoid brush is a linear polymer chain comprising one or more functional groups at one end where the functional group may be a hydroxyl (—OH), primary amine (—NH₂), thiol (—SH), carboxylic acid (—COOH), or dihydroxyphenylalanine (DOPA) functional group.

11. The device of claim 9, wherein the polypeptoid brush is a linear polymer chain comprising a single functional group at the other end where the functional group may be a biotin, alkyne/azide/thiol/ene, or RGD (Arg-Gly-Asp) peptide.

12. The device of claim 9, wherein the polypeptoid brushes are monodisperse, sequence-defined polymer chains, with each monomer of the polymer chain precisely defined.

13. A patterned structure comprising a monolayer of correspondingly functionalized polypeptoid molecules tethered to selected patterned areas, producing regions with surface passivation and regions with active binding properties.

14. A method to produce regions that alternate between passivated and non-passivated areas on a device, the method comprising: (a) providing the device of claim 2 comprising a patterned structure comprising the monolayer of the PMA; (b) lithographic patterning the device, (c) removing exposed portions of the PMMA brush not covered by the lithographic resist, and (d) removing the lithographic resist to produce regions that alternate between passivated and non-passivated areas against adsorption of nucleic acid nanostructures.

15. The method of claim 14, wherein the lithographic patterning step comprises optical, electron-beam, nano-imprint, or soft lithography.

16. A method to produce regions that alternate between areas with two different polypeptoid brushes on a device, the method comprising: (a) providing the device of claim 9 comprising a patterned structure comprising the monolayer of polypeptoid; (b) lithographic patterning the device; (b) removing the exposed portions of the polypeptoid brush not covered by the lithographic resist; (c) removing the lithographic resist to produce regions that alternate between areas with and without tethered polypeptoid brushes; and (d) tethering a second type of functionalized polypeptoids to the exposed regions to form a patterned structure with two different polypeptoid brushes tethered to selected areas.

17. The method of claim 16, wherein the lithographic patterning step comprises optical, electron-beam, nano-imprint, or soft lithography.
